(19) United States
(12) Patent Application Publication
(10) Pub. No.: US 2026/0216501 A1
(43) Pub. Date: Jul. 30, 2026

(54) Methods and Systems for Wireless Neuromodulation

(71) Applicant: Nia Therapeutics, Inc., Radnor, PA (US)

(72) Inventors: Daniel S. Rizzuto, Boston, MA (US); Zhe Hu, Boston, MA (US); Daniel Utin, Boston, MA (US); Joshua Kahn, Boston, MA (US); Chris Ho, Boston, MA (US); Andrew Smiles, Boston, MA (US); Michael J. Kahana, Boston, MA (US)

(21) Appl. No.: 19/462,651

(22) Filed: Jan. 28, 2026

Related U.S. Application Data
(60) Provisional application No. 63/750,464, filed on Jan. 28, 2025.

(51) Int. Cl. A61N 1/05; A61N 1/36; G16H 20/30; G16H 40/67

(52) U.S. Cl. CPC A61N 1/0529; A61N 1/36132; A61N 1/36189; G16H 20/30; G16H 40/67

(57) ABSTRACT

This application is directed to a neuromodulation system applying a closed-loop neural recording and stimulation process to treat cognitive or neurological dysfunctions. The neuromodulation system includes one or more electrode arrays implanted in a brain region of a patient, an implantable electronics package mounted on a skull under the scalp, and an external electronic system wearable by the patient. A plurality of channels are provided by a plurality of electrodes of the electrode arrays. Local field potential (LFP) recordings are collected from the brain region of the patient via the channels provided by the electrode arrays. The electronics package generates a spectral decomposition of the LFP recordings, updates a cognitive computational model, and delivers stimulation to the brain region of the patient via at least a subset of electrode arrays based on the cognitive computational model. The external electronic system enable at least wireless data communication with the electronics package.

### RELATED APPLICATION

This application claim benefit to U.S. Provisional Patent Application No. 63/750,464, filed Jan. 28, 2025, titled “Methods and Systems for Wireless Neurostimulation,” each of which is incorporated by reference in its entirety.

### GOVERNMENT RIGHTS

The subject matter of the invention may be subject to U.S. Government Rights under The Department of Defense grants: MTEC-20-06-MOM-013 from the Army Medical Research and Development Command.

### TECHNICAL FIELD

The present invention generally relates to cognitive restoration, including, but not limited to, methods and systems for neural recording and memory restoration using wireless implants.

### BACKGROUND

Memory loss constitutes one of the major health challenges affecting populations worldwide. Although public attention has largely focused on neurodegenerative diseases of memory, such as Alzheimer's disease, traumatic, infectious, and inflammatory insults to the brain can also cause profound memory loss in otherwise healthy individuals. These latter individuals do not benefit from pharmaceutical interventions that slow the progression of neurodegenerative disorders of memory such as lecanumab and donanemab.

Given the profound unmet need facing patients with memory deficits related to acquired brain injury, researchers have investigated electrical stimulation as an alternative therapeutic pathway. Several such studies have obtained promising results. In particular, stimulating lateral temporal cortex during predicted memory lapses produces reliable improvements in verbal episodic memory, especially when applying stimulation pulses near white matter tracts with strong functional connectivity to the broader memory network. In these studies, researchers used machine learning models to predict momentary lapses of memory encoding, triggering trains of high-frequency (100-200 Hz) stimulation during the predicted lapses.

Technological limitations, however, precluded deploying these therapies chronically, as in people have a device implanted in their brain that provides the therapy all the time. The machine learning algorithms require multi-electrode recordings from widespread brain regions and the use of these recordings to control delivery of stimulation to the brain in a closed-loop system. The aforementioned studies thus relied on the acute evaluation of the algorithms in patients undergoing neurosurgical evaluation for the treatment of drug-resistant epilepsy. However, one can only achieve proof of concept in these research studies.

### SUMMARY

In accordance with some embodiments of this application is at least a realization that electrical stimulation of the human brain has emerged as a powerful therapeutic modality, enabling the alteration of neural circuits underlying cognition and behavior. Further, in accordance with some embodiments of this application is at least a realization that stimulation's effects on physiology and behavior depend on endogenous variation in brain state, as measured by local field potential (LFP) recordings, and that combined hardware and software systems that can decode mnemonic variability and trigger stimulation chronically during everyday life are required to determine whether closed-loop stimulation constitutes a viable chronic therapy for individuals suffering from memory loss.

Various embodiments of this application are directed to integrating a plurality of channels in a wireless brain-computer interface (also called a Smart Neurostimulation System (SNS)) for recording local neural activities and adaptively stimulating a targeted neural area. In some embodiments, artificial intelligence (AI) processes neural signals collected from the neural activities and determine a stimulation for a targeted neural area based on the neural activities. In some embodiments, the SNS combines closed-loop analysis of spectral features of the field potential with multi-channel stimulation capabilities, and measures neural correlates of behavior (motion) and the physiological effects of stimulation. In an example, the SNS safely stimulated the brain, as determined through histology of brain tissue conducted following 120 days of stimulation.

In one aspect, a neuromodulation system is applied to treat cognitive or neurological dysfunctions. The neuromodulation system includes a plurality of electrode arrays implantable in a brain region of the patient, a skull-mounted implantable electronics package coupled to the plurality of electrode arrays, an external electronic system wearable by the patient, and configured for enabling at least wireless data communication with the implantable package, and a cloud-based software system that personalizes the computational model for each patient. The electronics package includes one or more processors and memory storing one or more programs for execution by the one or more processors. The one or more programs include instructions for embedding a computational model for memory enhancement, concurrently collecting LFP recordings from the patient via a plurality of channels located on the plurality of electrode arrays, updating the cognitive computational model based on the spectral decomposition of the LFP recordings, and delivering stimulation to the patient in which the electronics package has been implanted, including stimulating, via at least a subset of the plurality of electrode arrays.

In some embodiments, power and data telemetry are provided to the implantable package using a radio frequency (RF) coil system. In some embodiments, the plurality of channels includes 64 channels. In some embodiments, the skull-mounted implantable electronics package further includes a hermetically packaged battery-less electronics module for recording and stimulation, one or more coaxial RF coils for power and data telemetry, and a probe implanted in the brain region. In some embodiments, the patient is a human patient with one of a traumatic brain injury (TBI), Alzheimer's disease, epilepsy, major depression, attention-deficit hyperactivity disorder (ADHD), Parkinson's disease, and essential tremor. In some embodiments, the implantable electronics package obtains the cognitive computational model from an external server via a communications module in the electronics package. In some embodiments, the external electronic system is configured to generate a plurality of predefined stimulation patterns based on the cognitive computational model. The stimulation is delivered based on the plurality of predefined stimulation patterns. In some embodiments, delivering stimulation includes activating multiple brain areas associated with memory performance. In some embodiments, the local field potential recordings are processed prior to transmission to an external electronic system. In some embodiments, stimulation is delivered to at least one of a temporal lobe, a frontal lobe, a middle temporal gyrus, a left parietal lobe, a hippocampus, an entorhinal cortex, and a right parietal lobe of the brain region.

In yet another aspect, a method is implemented at a neuromodulation system for treating cognitive or neurological dysfunctions. The system includes one or more electrode arrays, an implantable electronics package, and an external electronic system. The method includes identifying a plurality of channels provided by a plurality of electrodes of the one or more electrode arrays, and the one or more electrode arrays are implanted in a brain region of a patient. The method further includes collecting LFP recordings from the brain region of the patient via the plurality of channels provided by the one or more electrode arrays; generating a spectral decomposition of the LFP recordings by the implantable electronics package, the implantable electronics package is mounted on a skull under the scalp; updating, by the implantable electronics package, a cognitive computational model based on the spectral decomposition of the LFP recordings; based on the cognitive computational model, delivering stimulation to the brain region of the patient via at least a subset of the one or more electrode arrays; and enabling, by an external electronic system, wireless data communication with the implantable electronics package.

Some implementations of this application include a system that includes one or more processors and memory having instructions stored thereon, which when executed by the one or more processors cause the one or more processors to perform any of the above methods.

Some implementations include a non-transitory computer readable storage medium storing one or more programs. The one or more programs include instructions, which when executed by one or more processors cause the processors to perform any of the above methods.

In some implementations, a neuromodulation system is configured to enable simultaneous 60-channel brain activity monitoring and automatically configurable, targeted electrical stimulation based on real-time spectral analysis of neural data. In some embodiments, the neuromodulation system is configured to modulate alpha-band power in response to stimulation. The neuromodulation system is configured to decode behavioral states from neural signals and predict motor activity. In some embodiments, electrodes of the neuromodulation system are configured to provide a plurality of channel schemes (e.g., a single electrode scheme involving a single electrode, a bipolar scheme involve two electrodes, a multi-electrode scheme involving a plurality of electrodes (e.g., 4 electrodes, 8 electrodes)) for neural recording and stimulation. Further, in some situations, the neuromodulation system provides a spatial resolution better than a spatial limit (e.g., 50 μm) and a temporal resolution better than a temporal limit (e.g., 2 ms) for neural recording and stimulation using at least one of the plurality of channel schemes. For example, the spatial resolution is 20 μm and the temporal resolution is 100 μs.

These illustrative embodiments and implementations are mentioned not to limit or define the disclosure, but to provide examples to aid understanding thereof. Additional embodiments are discussed in the Detailed Description, and further description is provided there.

### BRIEF DESCRIPTION OF THE DRAWINGS

For a better understanding of the various described implementations, reference should be made to the Detailed Description below, in conjunction with the following drawings in which like reference numerals refer to corresponding parts throughout the figures.

FIG. 1 is a flow diagram illustrating an example process of applying a smart neurostimulation system (SNS), in accordance with some embodiments.

FIGS. 2A and 2B illustrate example prototype of an example SNS, in accordance with some embodiments.

FIG. 3 is a block diagram illustrating an implementation of a cloud-based neuromodulation system including an SNS, in accordance with some embodiments.

FIG. 4A is a diagram showing main components of a brain, and FIG. 4B is a diagram showing subcortical ROIs, in accordance with some embodiments.

FIG. 5 is a block diagram of an example SNS, in accordance with some embodiments.

FIG. 6 is a flow diagram of an example neural modulation process implemented to record neural activities and deliver neural stimulations, in accordance with some embodiments.

FIG. 7A is a flow diagram of an example preclinical study process, in accordance with some embodiments, and FIG. 7B illustrate an example surgical procedure for chronic animal testing, in accordance with some embodiments.

FIG. 8 are temporal diagrams of LFP recordings, in accordance with some embodiments.

FIG. 9 are diagrams of example alpha power changes of six bipolar electrode pairs, in accordance with some embodiments.

FIG. 10 shows a temporal diagram of a classifier output, a correlation curve between a hit rate and a false alarm rate, a heatmap associated with bipolar electrode pairs, and temporal diagrams of two spectral powers and, in accordance with some embodiments.

FIG. 11A is a block diagram of an example SNS, in accordance with some embodiments.

FIG. 11B is a block diagram of a computer system that collaborates with the SNS to provide a cloud-based neuromodulation platform, in accordance with some embodiments.

FIG. 12 is a flow diagram of an example method of implementing closed-loop neural stimulation for memory restoration, in accordance with some embodiments.

FIG. 13A is a table including animal characteristics and depth leads implanted into left and right hemispheres, in accordance with some embodiments.

FIG. 13B is a table comparing characteristics of six example devices, in accordance with some embodiments.

FIG. 13C is a table showing anatomical locations for a plurality of monopolar electrodes, in some embodiments.

FIG. 14 is a set of example diagrams for classifying animal movement, in some embodiments.

FIG. 15 is a set of example diagram showing results of parameter search, in accordance with some embodiments.

FIG. 16 is a set of example diagrams showing stimulation's physiological effects, in some embodiments.

FIGS. 17 and 18 illustrate site-to-site heterogeneity in effects of stimulation amplitude, frequency, and duration on alpha and gamma, in accordance with some embodiments.

FIG. 19 illustrates linear mixed effects modeling of stimulation parameters, in accordance with some embodiments.

FIG. 20 illustrates that stimulation the therapy parameter increased alpha-power in closed-loop, in accordance with some embodiments.

The foregoing summary, as well as the following detailed description of embodiments of the system and method for virtual-assistant-enhanced access of private information, will be better understood when read in conjunction with the appended drawings of an exemplary embodiment. It should be understood, however, that the invention is not limited to the precise arrangements and instrumentalities shown.

Like reference numerals refer to corresponding parts throughout the several views of the drawings.

### DETAILED DESCRIPTION

Reference will now be made in detail to specific embodiments, examples of which are illustrated in the accompanying drawings. In the following detailed description, numerous non-limiting specific details are set forth in order to assist in understanding the subject matter presented herein. But it will be apparent to one of ordinary skill in the art that various alternatives may be used without departing from the scope of claims and the subject matter may be practiced without these specific details. For example, it will be apparent to one of ordinary skill in the art that the subject matter presented herein can be implemented on many types of electronic devices with digital video capabilities.

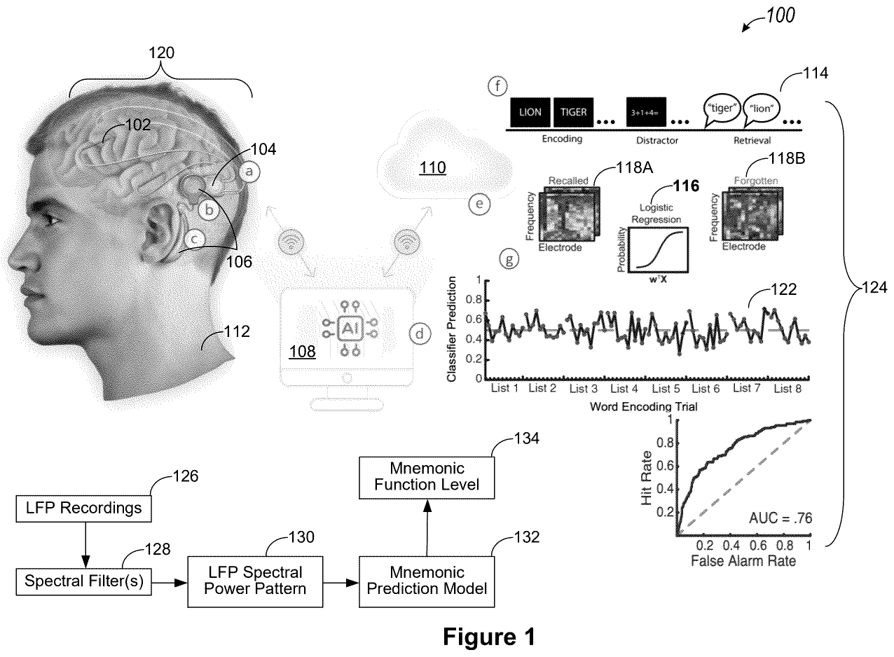

FIG. 1 is a flow diagram illustrating an example process 100 of applying a smart neurostimulation system (SNS) 120, in accordance with some embodiments. In some embodiments, the SNS 120 includes one or more electrode arrays 102, an implantable electronics package 104, and an external electronic system 106. The SNS 120 is coupled to a computer device 108 and a server 110 to form a cloud-based neuromodulation system for recording neural activities and stimulating selected brain regions, e.g., in a closed-loop manner. In an example, the one or more electrode arrays 102 of the SNS 120 include four depth leads with 64 contacts for sensing and stimulating neural tissue. In some embodiments, the implantable electronics package 104 includes a cranially-mounted implantable pulse generator (IPG) for processing neural data and controlling neural stimulation, and the external electronic system 106 includes a processor and is configured to provide power and act as a communications hub for the SNS 120. For example, the implantable electronics package 104 may be configured to harvest RF power from its surroundings by itself, and the power may be provided via an inductive link between the external electronic system 106 and the implantable electronics package 104. The implantable electronics package 104 may have a rechargeable configuration (e.g., include a battery) for storing power received from the external electronic system 106 and/or harvested by itself. In some embodiments, the computer device 108 is coupled to the external electronic system 106 of the SNS 120, and configured to execute a clinician program for task administration and therapy programming. Examples of the computer device 108 include, but are not limited to, a mobile phone, a desktop computer, a laptop computer, a dedicated personal device, and a tablet computer. The server 110 is coupled to the SNS 120 via the computer device 108, and configured to host a cloud-based AI platform having a plurality of accounts to personalize neural therapy for a plurality of patients 112.

In some embodiments, the SNS 120 configured to predict momentary memory lapses based on a plurality of LFP recordings 126 (operation 114) of multiple electrodes in widespread regions while the patient 112 studies and recalls word lists. A machine learning model (e.g., one or more multivariate classifiers 116) may be applied to learn a mapping between a distribution 118A or 118B of spectral power across electrodes and a subsequent recall status 122 of encoded items. An example of the multivariate classifiers 116 is a penalized logistic regression (e.g. based on L2). In some embodiments, the distribution 118, the subsequent recall status 122, and the mapping form a cognitive computational model 124 measured for the patent 112, and provide stimulation data (e.g., stimulation parameters and their timing) that define stimulations delivered to restore memory of the patient 112.

More specifically, in some embodiments, the patient 112 performs repeated memory tasks in which they study and subsequently recall lists of common words, which is a standard method used in neuropsychological assessments of memory function). The SNS 120 records multi-channel field potentials during each phase of the memory task. One or more spectral filters 128 are applied to a time series of LFP recordings 126 (also called electroencephalogram (EEG) data), allowing the IPG of the implantable electronics package 104 to estimate an LFP spectral power pattern 130 at a frequence range (e.g., including frequencies ranging from 3 to 180 Hz). In some embodiments, a machine learning model (e.g., a mnemonic prediction model 132) is trained to process the LFP spectral power pattern 130 and predict a level of a mnemonic function 134 using the pattern of spectral power estimated across frequencies and channels. In some embodiments, the machine learning model is applied to process LFP recordings 126 measured from unseen holdout sessions and determines how well it can predict variability in memory performance, providing a rapid readout of mnemonic function 134 at any given time. In an example, the machine learning model is applied to identify or predict a memory lapse in real time while the LFP recordings 126 are collected. In accordance with an identification or prediction of the memory lapse, the SNS 120 may identify a target brain region based on the LFP recordings 126 and deliver a stimulation in the target brain region to restore memory for the patient 112.

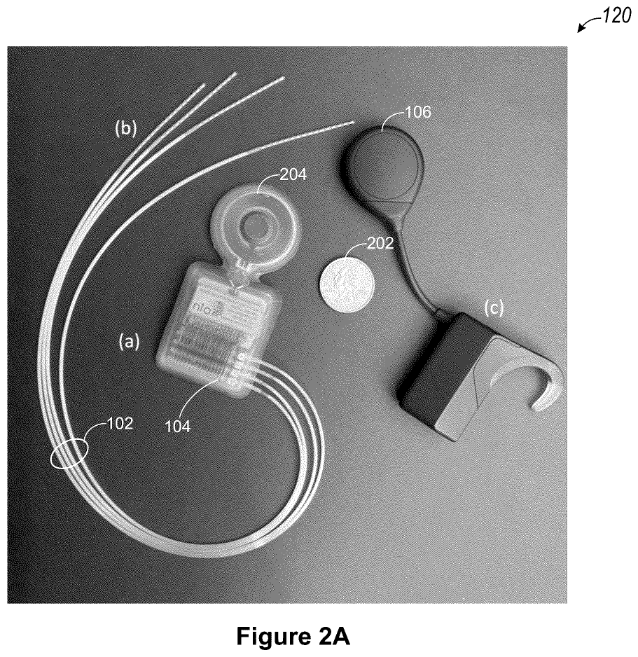

FIG. 2 illustrates a prototype of an example SNS 120, in accordance with some embodiments. The example SNS 120 includes one or more electrode arrays 102 (e.g., four depth leads containing up to 16 electrodes each), an implantable electronics package 104 (e.g., an implantable pulse generator (IPG)), and an external electronic system 106 (e.g., including an external processor (EP)). Device materials of components of the SNS 120 are selected for biocompatibility and history of usage within medical devices based on a respective location of each component. In an example, the IPG of the implantable electronics package 104 is fully encapsulated in silicone, and includes embedded electronics that are enclosed in a hermetically-sealed titanium can. The one or more electrode arrays 102 are packaged in a lead connector assembly that further includes platinum/iridium, stainless steel, and pellethane. The implantable electronics package 104 further includes a gold-coated magnet coated with parylene-C. In some embodiments, a depth lead of the one or more electrode arrays 102 includes platinum/iridium electrodes, a stainless-steel set screw block, and a pellethane lead body. Materials used in the external electronic system 106 include acrylonitrile butadiene styrene (ABS) plastic, nylon, and silicone. A quarter 202 is included for scale.

In some embodiments, the IPG senses local field activity from 60 electrodes and 4 reference electrodes, processes resulting neural data (e.g., LFP recordings 126 in FIG. 1), and controls therapy delivery. In some embodiments, the EP contains a rechargeable battery and provides power to the IPG via an inductive link 204. The inductive link 204 between the IPG and the EP has a 2.2 Mbps data rate, which provides a sufficient data bandwidth to stream 1000 Hz LFP data from all 60 electrodes to the EP of the electronic system 106. A reliable data transmission distance across scalp distances ranges from 4 to 10 mm. In some embodiments, the EP acts as a communication hub between the SNS 120 and the computer device 108, e.g., by providing communication with a computer device 108 via a Universal Serial Bus (USB) link or a Bluetooth Low Energy (LE) link. In some embodiments, the SNS 120 authenticates and encrypts all communications end-to-end using 128-bit AES-GCM, ensuring only authenticated users and devices can connect to the device and preventing administration of malicious therapy parameters. In some embodiments, the EP is intended to be recharged overnight, when therapy delivery is not required.

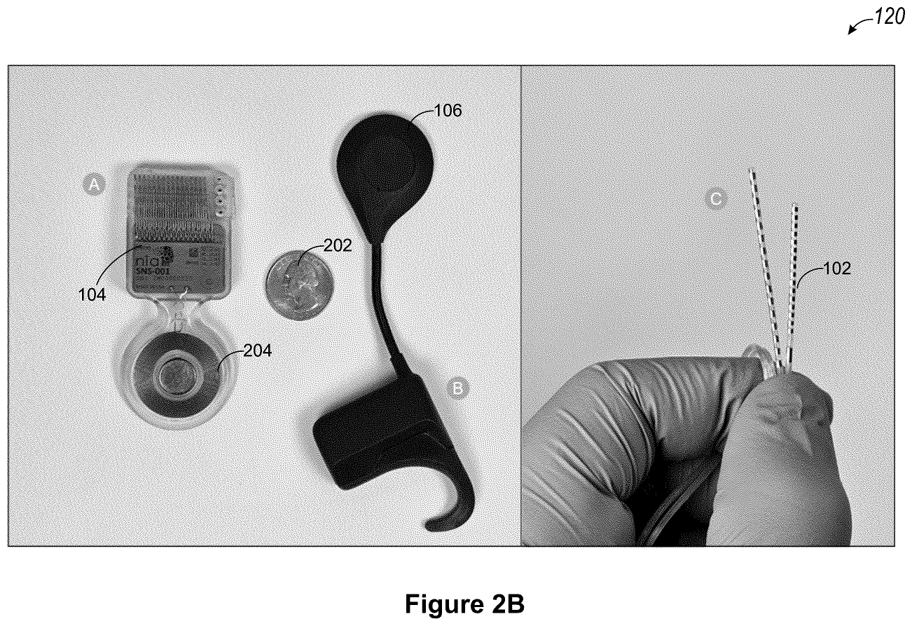

FIG. 3 is a block diagram illustrating an implementation of a cloud-based neuromodulation system 300 including an SNS 120, in accordance with some embodiments. While some example features are illustrated, various other features have not been illustrated for the sake of brevity and so as not to obscure pertinent aspects of the example embodiments disclosed herein. To that end, as a non-limiting example, the cloud-based neuromodulation system 300, referred to herein as system 300, may include an SNS 120 and one or more computer devices 108 in communication with a networked server 110.

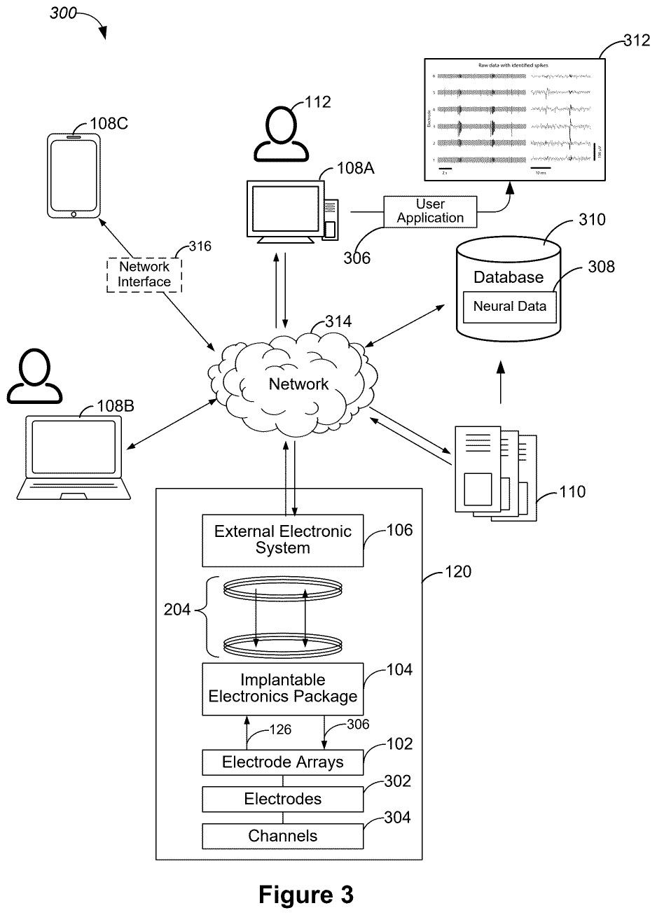

The SNS 120 includes one or more electrode arrays 102 including a plurality of electrodes 302, an implantable electronics package 104 coupled to the one or more electrode arrays 102, and an external electronic system 106 wearable by a patient 112. The one or more electrode arrays 102 are configured to be implanted in a brain region (e.g., temporal lobe 410 in FIG. 4A) of the patient 112 and provide a plurality of channels 304 based on the plurality of electrodes 302. The electronics package 104 is configured to be mounted on a skull under the scalp, and includes one or more processors and memory storing a cognitive computational model for memory enhancement and one or more programs for execution by the one or more processors. In some embodiments, the electronics package 104 concurrently collects LFP recordings 126 (FIG. 1) from the brain region of the patient 112 via the plurality of channels 304 provided by the one or more electrode arrays 102, generates a spectral decomposition of the LFP recordings 126, and updates a cognitive computational model 124 of the patient 112 based on the spectral decomposition of the LFP recordings 126. Based on the cognitive computational model 124, the electronics package 104 identities a brain state of the patient 112 (e.g., whether a memory lapse occurred or will occur to a particular brain region), and delivers stimulation to the brain region of the patient 112 via at least a subset of the one or more electrode arrays 102 (e.g., corresponding to the particular brain region). The external electronic system 106 is configured to provide power and data telemetry to the implantable electronics package 104 wirelessly.

In some embodiments, the system 300 includes the server 110, one or more computer devices 108, and one or more databases 310. The server 110 and the computer device 108 may execute a respective user application 306 for obtaining LFP recordings 126 and associated neural data 308, facilitating delivery of neural stimulations, and providing secure access to neural data 308 (e.g., LFP data and stimulation data) which may be stored on a database 310. More specifically, in some embodiments, the server 110 executes a server-side user application 306, and the computer device 108 executes a client-side user application 306. Further, in some embodiments, each of the external electronic system 106 and the implantable electronics package 104 executes a local user application associated with the user application 306 to facilitate collection of LFP recordings 126 and delivery of neural stimulations. In some embodiments, the user application 306 is configured to render a graphical user interface 312 for display on the computer device 108, thereby visualizing the LFP recordings 126, the cognitive computational model 124, or a neural stimulation pattern associated with a neural stimulation.

In some embodiments, machine learning models are applied by the SNS 120, the computer device 108, and/or the server 110 to process LFP recordings 126 or generate neural stimulation. For example, one or more multivariate classifiers 116 may be applied to process the LFP recordings 126 and learn a mapping between a distribution 118A or 118B of spectral power across electrodes and a subsequent recall status 122 of encoded items. In another example, a mnemonic prediction model 132 is applied to process a LFP spectral power pattern of the LFP recordings 126 and predict a level of a mnemonic function 134. In some implementations, a machine learning model is trained or fine-tuned at the server 110 and deployed to the computer device 108 or the SNS 120 for execution. In some implementations, a machine learning model is trained at the server 110 and deployed to the computer device 108 or the SNS 120 for fine-tuning and/or execution.

The one or more computer devices 108 may be, for example, desktop computers 108A, laptop computers 108B, mobile phones 108C, tablet computers, or any other computing devices. Each computer device 108 can collect data or user inputs, executes the client-side user application 306, and present outputs (e.g., LFP recording 126, stimulation data) on its user interface 312. The collected data or user inputs can be processed locally at the computer device 108 and/or remotely by the server(s) 110. The server 110 provides system data (e.g., boot files, operating system images, and user applications) to the computer devices 108, and in some embodiments, processes the data and user inputs received from the computer device(s) 108 when the user application 306 is executed on the computer devices 108, e.g., associated with different patients 112 or medical practitioners. In some embodiments, the database 310 stores data related to the server 110, computer devices 108, user applications 306, and their associated SNS 120.

In some embodiments, the implantable electronics package 104 is wirelessly coupled to the external electronic system 106 via an inductive link 204 within the SNS 120. Conversely, in some embodiments, the server 110, one or more computer devices 108 (e.g., devices 108A-108C), database 310, and external electronic system 106 of the SNS 120 are communicatively coupled to each other via one or more communication networks 314, which are the medium used to provide communications links between these devices and computers connected together within the system 300. The one or more communication networks 314 may include connections, such as wire, wireless communication links, or fiber optic cables. Examples of the one or more communication networks 314 include local area networks (LAN), wide area networks (WAN) such as the Internet, or a combination thereof. The one or more communication networks 314 are, optionally, implemented using any known network protocol, including various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol. A connection to the one or more communication networks 314 may be established either directly (e.g., using 3G/4G connectivity to a wireless carrier), or through a network interface 316 (e.g., a router, switch, gateway, hub, or an intelligent, dedicated whole-home control node), or through any combination thereof. As such, the one or more communication networks 314 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages.

The server 110 is configured to enable real-time data communication with the computer devices 108 that are remote from each other or from the server 110. In some embodiments, the server 110 is configured to communicate with the SNS 120 via the computer device 108. Alternatively, in some embodiments, the server 110 is configured to communicate with the SNS 120 via the communication network 314 without involving the computer device 108. Further, in some embodiments, the server 110 is configured to implement data processing tasks that cannot be or are preferably not completed locally by the computer devices 108 or the SNS 120. For example, a computer device 108 includes a mobile phone 108C that applies machine learning models having sizes not executable on the mobile phone 108C. In some embodiments, these machine learning models are created based on one or more neural networks to process the LFP recordings 126 or generate neural stimulations. A machine learning model may be trained with training data before they are applied for neural recording or stimulation.

FIG. 4A is a diagram showing main components of a brain 400, and FIG. 4B is a diagram showing subcortical ROIs 450, in accordance with some embodiments. The brain 400 includes frontal lobes 402, parietal lobes 404, occipital lobes 406, cerebellum 408, temporal lobes 410, and a brain stem 412. The temporal lobes 410 are located on the sides of your head, roughly around the area of your temples and behind ears. The temporal lobes 410 are used for processing auditory information and encoding memory. In some embodiments, the temporal lobes 410 are configured to process affect and/or emotions, language, and certain aspects of visual perception. In some embodiments, a dominant temporal lobe is involved in understanding language and learning and remembering verbal information. A non-dominant lobe is involved in learning and remembering non-verbal information (e.g. visuo-spatial material and music). The temporal lobes 410 is key for sensory perception, memory, emotional responses, and language comprehension. One or more electrode arrays may be implanted into at least one functional part 402-412 of the brain 400. In some embodiments, the SNS 120 is configured to record signals from a plurality of distributed regions of a brain (e.g., a cortex) via electrodes of the electrode array(s). Based on configurations of the electrodes, the distributed regions may cover a small or large portion of the brain as needed.

Referring to FIG. 4B, the subcortical ROIs 450 of the brain 400 include amygdala (Amy), hippocampus (Hip), pallidum (PA), and putamen (PU). The hippocampus (Hip) and the amygdala (Amy) are located within the temporal lobe 410 and are associated with memory, emotions, and learning. The hippocampus (Hip) is involved in forming new memories, and the amygdala (Amy) is involved in processing emotions, particularly fear and pleasure. In some embodiments, the one or more electrode arrays 102 are implanted into the subcortical ROIs 450 of the brain 400, and are configured to stimulate the subcortical ROIs 450, e.g., to facilitate memory restoration for a patient.

Conversely, in some embodiments not shown, the one or more electrode arrays 102 are implanted into cortical ROIs of the brain 400, and are configured to stimulate cortical ROIs, e.g., to facilitate memory restoration for a patient.

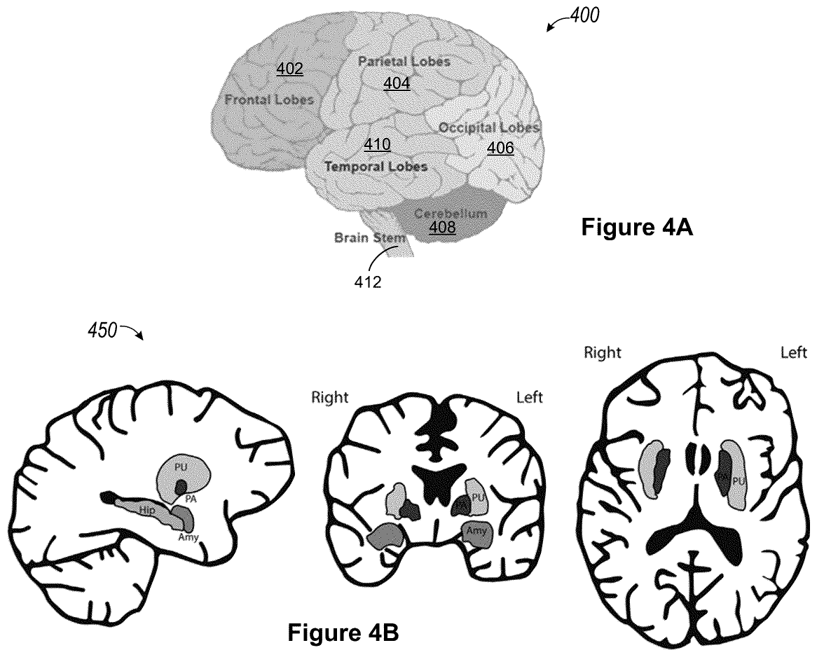

FIG. 5 is a block diagram of an example SNS 120, in accordance with some embodiments. The example SNS 120 includes one or more electrode arrays 102 (e.g., four depth leads containing up to 16 electrodes each), an implantable electronics package 104 (e.g., an implantable pulse generator (IPG) 502), and an external electronic system 106 (e.g., including an external processor (EP) 504). The EP 504 and the IPG 502 interact via an inductive link 204 including two conductive coils, allowing wireless power and data transmission. Command and response data flow bidirectionally through this inductive link 204, while neural data are transmitted in parallel from the IPG 502 to the EP 504. In some embodiments, neural data are received by the EP 504 and further sent to the computer device 108 via a Universal Serial Bus (USB) link or a Bluetooth Low Energy (LE) link.

In some embodiments, the EP 504 further includes a microcontroller unit (MCU) 508 configured to control system logics (e.g., for communication, safety management, and firmware update). In some embodiments, the EP 504 includes a field programmable gate array (FPGA) 531, and the IPG 502 includes an FPGA 509. The FPGAs 509 and 531 are configured to apply a communication protocol and enable communication over the inductive link 204. On the IPG 502, power is received over the inductive link 204 and regulated by power management hardware 512 (e.g., power management integrated circuit (PMIC)), while the MCU 514 is configured to process communication messages received from the EP 504 and manage therapy safety during neural stimulation.

In some embodiments, the IPG 502 enables an electrical interface 516 to neural tissue. The electrical interface 516 includes an application specific integrated circuit included in the IPG 502 and an IPG lead interface 520 that holds one or more electrode arrays 102 having a plurality of electrodes 302 (e.g., four leads with 16 electrodes each). In an example, the electrode arrays 102 include 64 Platinum-Iridium electrodes, in which 60 electrodes are configured to sense and stimulate neural tissue and remaining four electrodes, one arranged on each lead, serve as electrical references for the electrode arrays 102. In some embodiments, the ASIC includes a neuromodulation integrated circuit (NMIC) 518, and an output of the NMIC 518 is electrically coupled to, and configured to drive, the electrodes 302 of the electrode arrays 102, e.g., via DC-blocking capacitors. In some embodiments, the plurality of electrodes 302 are configured to provide a plurality of channels 304, and the NMIC 518 is configured to collect LFP recordings 126 from, and deliver stimulation to, the plurality of channels 304 independently from one other. In an example, the NMIC 518 has 64 independent sensing and stimulation channels, one for each electrode 302. Alternatively, in some embodiments, the NMIC 518 has less than 64 independent sensing and stimulation channels, and each sensing and stimulation channel is applied to sense or stimulate a subset of the plurality of channels 304 sequentially. By these means, the NMIC 518 and the electrode arrays 102 collaborate with one another to provide a brain interface with sensing and stimulation capabilities.

In some embodiments, the SNS 120 includes a plurality of channels (e.g., 64 channels) including a first subset of reference channels acting as electrical references and a second subset of work channels for neural signal recording and stimulation. In an example, the SNS 120 includes 64 channels including 4 reference channels and 60 work channels.

In some embodiments, the IPG lead interface 520 is coupled between the brain and the NMIC 518, and includes an RC circuit 522 that can record neural activity. In some embodiments, the IPG 502 includes an IPG circuit board on which one or more filtering capacitors 524 are applied to shunt noise to ground contacts. The NMIC 518 further includes a plurality of sense channels 526 and a plurality of stimulation drivers 528. For sensing neural activities of the brain, each sense channel 526 is configured to collect an LFP recording 126 from a respective channel 304 of a respective electrode 302. In an example, the sense channels 526 continuously sample neural signals to generate LFP recordings 126 (e.g., at a sampling rate of 1000 Hz) with reference to GND contacts. In some embodiments, the NMIC 518 provides a copy of the LFP recordings 126 to a local FPGA 530 for local pre-processing and analysis, thereby generating neural data 532 associated with the LFP recordings 126. The IPG 502 transfers the LFP recordings 126 or the neural data 532 for the plurality of channels 304 of the plurality of electrodes 302 (e.g., at 1000 Hz) to the EP 504. The EP 504 receives the LFP recordings 126 or the neural data 532 via the inductive link 204, and may forward the LFP recordings 126 or the neural data 532 to the computer device 108 wirelessly or using a wire.

In some embodiments, for neural stimulation, the IPG 502 is configured to deliver a biphasic, square-wave, stimulation pulses 534 in a bipolar configuration through a set of electrodes 302 (e.g., two electrodes including an anodic electrode 304A and a cathodic electrode 304C) located on the same lead (also called electrode array 102). For example, during a first phase of a stimulation, a stimulation driver 528 sources a first stimulation current through the anodic electrode 304A and sinks the first stimulation current through the cathodic electrode 304C. During a second phase following the first phase, the stimulation driver 528 sources a second stimulation current through the cathodic electrode 304C and sinks the second stimulation current through the anodic electrode 304A. The first phase and the second phase of each stimulation pulse 534 may be symmetric or asymmetric to each other. In some embodiments, the electrodes 304A and 304C are separated by an integer number of electrodes, and the integer number is equal to 1, 2, 3, . . . , and N-3, where N is a total number of electrodes on a respective electrode array 102. In some embodiments, the electrodes 304A and 304C are immediately adjacent to each other on a respective electrode array 102. In another example, the set of electrodes 302 includes three electrodes (e.g., an anodic electrode 304A and two cathodic electrode 304C and 304B).

In some embodiments, the SNS 120 is configured to meet a plurality of performance constraints for one or more of a recording region size, an LFP sampling rate, stimulation parameters, a form factor, and other performance factors. For example, in accordance with the performance constraints, the SNS 120 collects LFP recordings 126 from >32 electrodes in widespread brain locations at sampling rates greater than or equal to 500 Hz (to discern high-frequency correlates of variable memory function). In some embodiments, in accordance with the performance constraints, the SNS 120 is configured to rapidly deliver bursts of electrical stimulation in response to predicted memory lapses, derived from machine learning models trained on data sampled from the device. Furthermore, In some embodiments, in accordance with the performance constraints, the SNS 120 includes a cranial implant of a similar size to those proven safe in a responsive neurostimulation (RNS), which is an implanted device that monitors brain waves for unusual patterns that indicate a seizure and automatically sends electrical pulses to interrupt seizure activity.

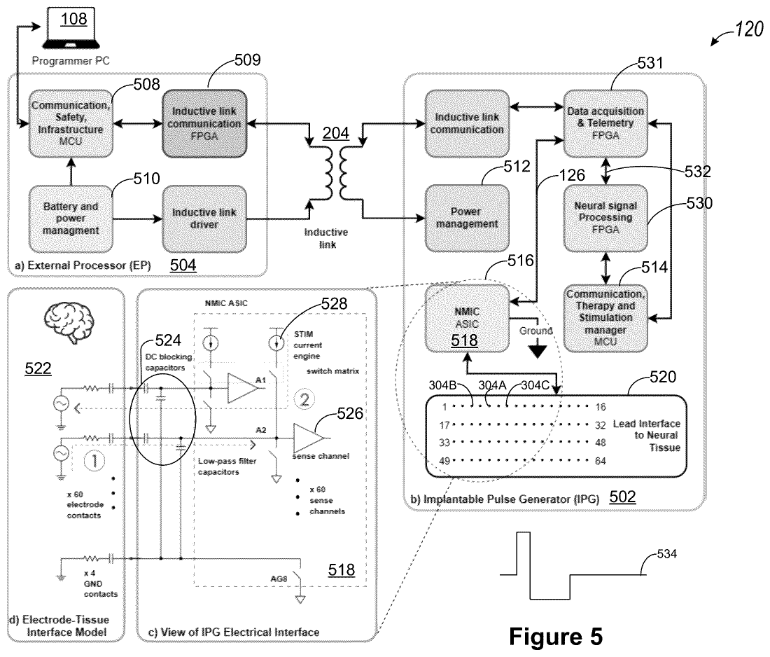

In some embodiments, the SNS 120 responsively stimulates neurons within 200 ms of a memory lapse event, which is detected by a multivariate classifier 116 based on LFP recordings 126 and/or neural data 308. The stimulation (e.g., stimulation 620 in FIG. 6) includes bursts of bi-phasic square wave pulses having frequencies between 50 and 200 Hz amplitudes of 0.1 to 3 mA, and durations of 250 ms to 1 second. In another example, the bi-phasic square wave pulses have frequencies in a greater frequency range (e.g., 15-256 Hz) and last for more than 1 second (e.g., for 2 seconds). Power requirements of the SNS 120 are determined based on stimulation parameters along with the requirements of recording using a 500 Hz sampling rate from the plurality of channels 304 and evaluating brain state 8 times per second. In some embodiments, the SNS 120 has a power consumption lower than 25 mW, which is provided by a battery 510 disposed in the external electronic system 106. In some embodiments, stimulations are delivered within a period of 12 hours between charges, requiring the battery 510 to have a capacity greater than 75 mAH. In some embodiments, the battery is replaced at a frequency lower than one every three years. The power applied to deliver stimulations are transmitted from the external electronic system 106 to the implantable electronics package 104 via an inductive link 204, introducing an inductive power loss. The battery 510 provides the power applied to deliver stimulations, the inductive power loss, and power usage of the external electronic system 106. For example, the battery 510 has a capacity of 315 mAh to support approximately 12 hours of use.

FIG. 6 is a flow diagram of an example neural modulation process 600 implemented to record neural activities and deliver neural stimulations, in accordance with some embodiments. The process 600 is implemented by the SNS 120 (e.g., by MCUs and FPGAs of the IPG 502 and the EP 504). Process 600 is, optionally, governed by instructions that are stored in a non-transitory computer readable storage medium and that are executed by the one or more processors (e.g., MCUs 508 and 514, FPGAs 509, 530, and 531) of IPG 502 and the EP 504. Each of the operations shown in FIG. 6 may correspond to instructions stored in the memory or computer readable storage medium. The computer readable storage medium may include a magnetic or optical disk storage device, solid state storage devices such as Flash memory, or other non-volatile memory device or devices. The computer readable instructions stored on the computer readable storage medium may include one or more of: source code, assembly language code, object code, or other instruction format that is interpreted by one or more processors. Some operations in the process 600 may be combined and/or the order of some operations may be changed.

In some embodiments, the SNS 120 includes firmware running on both the IPG 502 and the EP 504 to collect and process LFP recordings 126 and administrate electrical stimulations 620. The LFP recordings 126 are collected from the brain and processed to generate (operation 602) neural data 532, and the neural data 532 can take two paths including a streaming path 604 where the neural data 532 are sent to an external computer device 108 (e.g., via a USB or Bluetooth link) and a therapy delivery path 606 where the neural data 532 are processed (operation 608) and therapy is administered (operation 610) based on brain state classification 612. In some embodiments, brain state classification 612 is implemented based on machine learning. In some embodiments, therapy is administrated by applying a stimulation with configured stimulation parameters. Further, in some embodiments, brain state classification 612, therapy delivery 610, or both are programmable and adaptable.

Referring to FIG. 6, in some embodiments, firmware of the SNS 120 is implemented to sense brain activity and make therapy decisions. The firmware ensures proper functioning of the EP 504 and the IPG 502 and includes multiple programs running on the MCUs 508 and 514 and the FPGAs 509, 530, and 531. The firmware supports communication for command and control, battery management, firmware updates, event logging, and other features. Therapeutic tasks of the firmware include transmission of the neural data 532 and therapy application of the electrical stimulation 620. The firmware may be implemented to transmit neural data 532 from the IPG 502 through the EP 504 to an external computer device 108, supporting offline analysis and therapy personalization. More specifically, neural signals (e.g., LFP recordings 126) are collected and preprocessed to generate neural data 532 by the IPG 502. The LFP recordings 126 and/or neural data 532 are collected and forwarded (operation 614) over the inductive link 204 to the EP 504. In some embodiments, the EP 504 tags (operation 616) the neural data 532 and sends (operation 618) the neural data 532 and/or associated tagged data to the computer device 108 (e.g., via a USB link). The computer device 108 may conduct offline analysis on the neural data 532 and/or associated tagged data. In some embodiments, the data streaming path 604 extends to operations 614, 616, and 618, and is independent of therapy administration.

In some embodiments, the therapy delivery path 606 is implemented by the IPG 502, which senses neural data 532, classifies the brain's state, and decides whether to apply an electrical stimulation 620. In this therapy delivery path 606, the LFP recordings 126 and/or neural data 532 are periodically processed by the FPGAs 530 to estimate a spectral power within multiple frequency bands in a given time window (e.g., 120 ms), thereby providing an LFP spectral power pattern 130. The processed neural data are used by a brain state classifier (e.g., classifier 116, model 132) in the MCU 514 to make a therapy decision based on a configured set of stimulation parameters determined by individualized AI-based training. In some embodiments, a multivariate classifier 116 (e.g., a logistic regression) may be applied to determine a cognitive computational model 124 of the patient 112 including a mapping between a distribution 118A or 118B of spectral power across electrodes and a subsequent recall status 122 of encoded items, thereby determining whether a therapy should be applied. In some embodiments, a mnemonic prediction model 132 is applied to process an LFP spectral power pattern 130 to determine a mnemonic function level 134 for the stimulation 620. In some situations, when the IPG 502 applies electrical the stimulation 620 to the brain, the parameters of this stimulation 620 are determined based on the cognitive computational model 124 or the mnemonic function level 134 of the patient 112, and include target electrode identifications (e.g., selecting one or more stimulation channels), a pulse amplitude, a pulse width, and a stimulation duration. In some embodiments, the brain state classifier and the stimulation parameters are configured based on each individual patient 112.

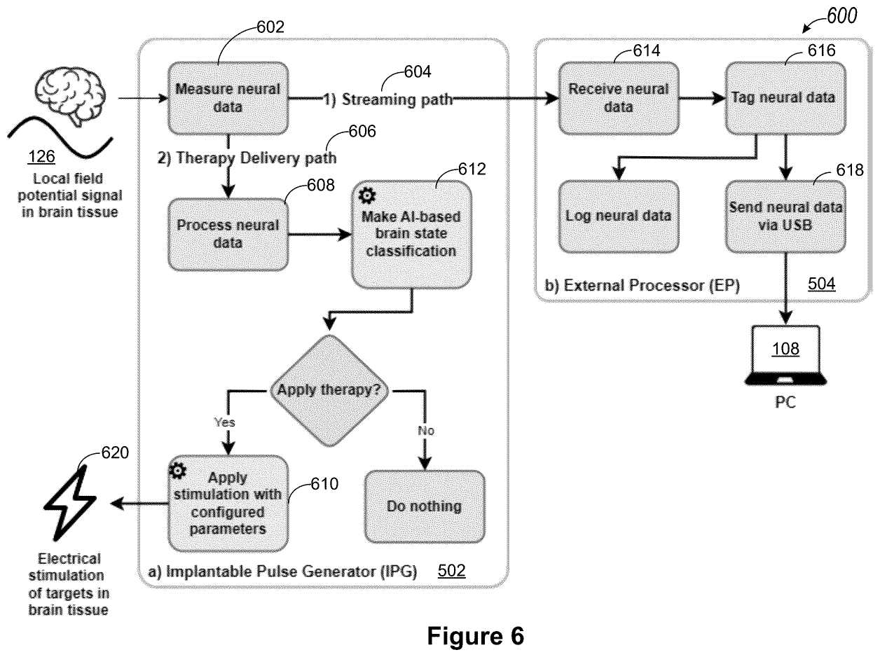

FIG. 7A is a flow diagram of an example preclinical study process 700, in accordance with some embodiments, and FIG. 7B illustrate an example surgical procedure 750 for chronic animal testing, in accordance with some embodiments. Computed tomography scans (CT scan) were collected on Day −7, Day 0 (post-implantation) and Day 120 (prior to study exit). An IPG 502 and a protective exoskeleton were implanted into a pocket in the neck, and two electrode arrays 102 (also called leads) were implanted into the left and right parietal lobes. The CT scans 702 show locations of control electrodes 704 implanted in right hemisphere. The CT scans 706 show locations of the electrodes 302 of the two electrode arrays 102 partially implanted in left hemisphere. In some embodiments, each of the two electrode arrays 102 includes 8 electrodes 302 configured to provide 8 channels 304, and is applied to record neural activity and apply brain stimulation (e.g., between Days 7 and 120 after the two electrode arrays 102 are implanted into an animal object), while the animal object moves freely. In some embodiments, histology may be performed to evaluate the effects of the device materials and electrical stimulation 620 on surrounding neural tissue.

In some situations, non-contrast computed tomography (CT) scans were collected during procedure planning (Day −7), following device implantation (Day 0), and prior to study exit (Day 20). Bright metal artifact on the post-surgical and study exit scans helped to visually identify and localize the electrodes. Excessive artifact creates diffuse signal that makes localization more difficult. In some embodiments, an iterative metal artifact rejection (iMAR) algorithm is applied during scanning to minimize signal diffusion.

In some embodiments, the electrode arrays 102 include an four-channel deep brain lead and a custom 8-channel lead utilizing industry-standard materials (e.g., Platinum/Iridium electrodes and pellethane lead body). In some embodiments, bipolar virtual electrode recordings are derived from the monopolar recordings. A midpoint of each pair of monopolar electrodes corresponds to a virtual electrode. In an example, two neighboring monopolar electrodes N1 and N2 are applied to provide a virtual electrode N1-N2 (also called a virtual bipolar electrode pair). In other words, virtual bipolar electrodes are created from the nearest neighbor monopolar electrodes. In another example, a virtual electrode is created by between the first electrode and the last electrode on each electrode array 102. In some embodiments, the SNS 120 samples multi-channel EEG signals at 500 Hz and generates charge-balanced square-wave stimulation pulses on any pair of electrodes with a range from 0.02-5 mA pulse amplitude, 15-500 μs pulse width, and 14.7-256 Hz pulse frequency. Stimulation trains may be unlimited in duration and may be bipolar-biphasic or monopolar-biphasic.

In some embodiments, the effect of brain stimulation may be evaluated on neural recordings. Stimulation parameters are varied across multiple sessions and days to evaluate the effect of stimulation amplitude and frequency on the neural signals recorded on nearby electrodes 302. In an example, 300 μs biphasic stimulation pulses are applied at frequencies of 25, 50, 100 and 200 Hz and amplitudes of 0, 200, 400, 600, 800, and 1000 μA across a bipolar pair of neighboring electrodes (e.g., N5-N6) located in left hemisphere white matter (e.g., corresponding to CT Scan 706 in FIG. 7B).

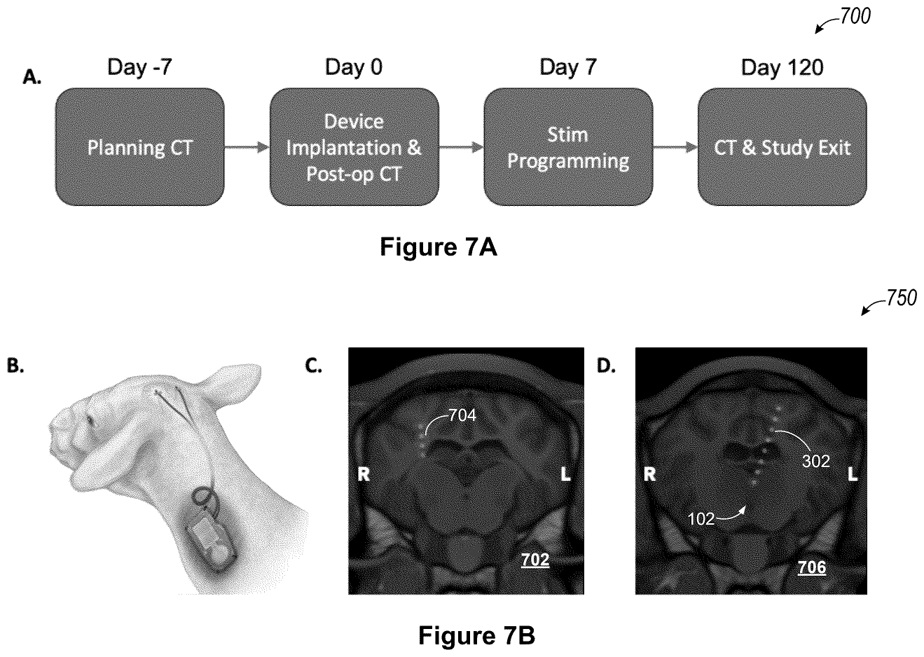

FIG. 8 are temporal diagrams of LFP recordings 126, in accordance with some embodiments. In some embodiments, 30 stimulation trials are applied, and each trial includes 24 frequency and amplitude combinations. An order of stimulation trials is shuffled to create 0 uniquely ordered session protocols that minimized trial order confounds. Each trial applies stimulation for 500 ms, with a random delay of 2000-2250 ms (uniformly distributed) between the offset and onset of consecutive stimulation trials. Overall, in some embodiments, a total of 41 sessions of 720 trials are collected, and each trail is collected from a single subject (e.g., for a total of 29,520 trials). Any trial window have temporal length less than 10 ms of data loss in each pre- or post-stimulation recording window.

In some embodiments, two electrodes 302 of two distinct electrode arrays 102 are electrically shorted to server as an electrical reference for electrode recordings of the two electrode arrays 102. Bipolar referencing, computed as the voltage difference between pairs of adjacent electrodes, filtered out signals common to both channels. This referencing scheme attenuates oculomotor and electromyographic artifacts that can mix with neural signals, and large-N human studies have shown that bipolar referencing of intraparenchymal electrodes better resolves the neural correlates of memory.

In some embodiments, inductive communication obviates the need for line noise filtration. Alternatively, in some embodiments, communication over the inductive link 204 can fail from momentary displacement of the coils. Communication failures lasting >10 ms may occur on approximately 1% of trials, which are identified and aborted. In some embodiments, an artifact arises as charge dissipates exponentially post-stimulation with a halftime of approximately 1000 ms. Spectral leakage from this decay has negligible impact on frequencies of interest in our analysis, which occur above 3 Hz.

In some embodiments, the effect of stimulation on neurophysiology is measured based on intracranial EEG signals recorded during the 750 ms preceding and following each stimulation train at each of six bipolar electrode pairs on the SNS lead. Based upon the fast-Fourier transform, the power spectral densities are calculated during the pre-stimulation and post-stimulation periods. A log-transform of each pre-stimulation power value 802 and post-stimulation power value 804 is determined with respect to a frequency in a frequency range (e.g., 0-80 Hz). For each frequency f, a difference 806 in post- and pre-stimulation power as ΔP(f)=P(f)post−P(f)pre is determined. In some embodiments, the SNS 120 can modulate post-stimulation neural activity. In some embodiments, a neuromodulatory index (e.g., the difference 806 pos-stimulation and pre-stimulation powers) reveals consistent changes for at least certain electrode frequency pairs.

In some embodiments, a stimulation 620 (e.g., having parameters of 200 Hz, 1 mA) is applied to increase high-frequency activity at nearby recording electrodes 302. In some embodiments, the difference 806 of pre-stimulation and post-stimulation spectral powers 802 and 804 varied as a function of stimulation amplitude and frequency at each bipolar electrode pair. For example, an increase of stimulation amplitude or stimulation frequency results in an increase of a high-frequency activity in a poststimulation period. In sham and some low amplitude conditions, decreases in alpha power are measured at a subset of the plurality of electrodes 302 of the electrode arrays 102. The curios decrease in power in the sham condition may reflect carryover effects from prior stimulation events.

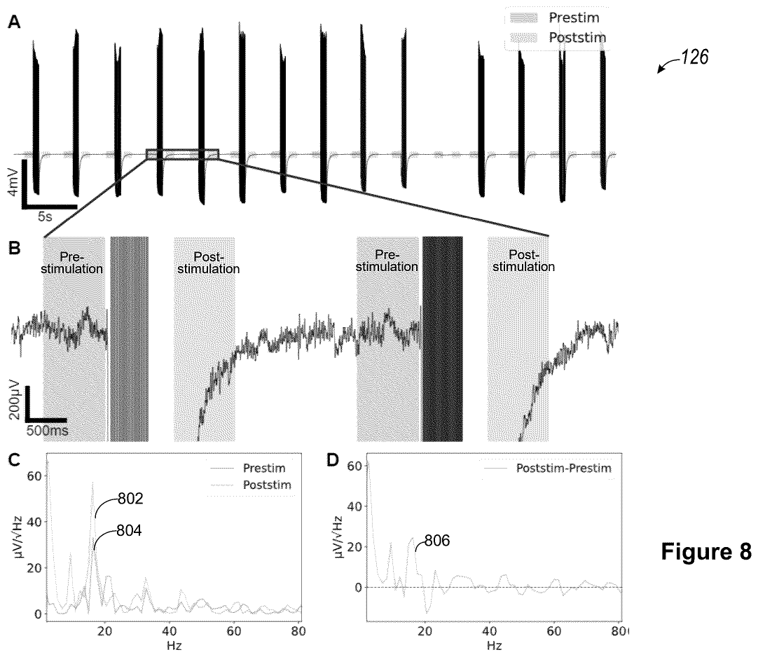

FIG. 9 are diagrams of example alpha power changes 900 of six bipolar electrode pairs 302, in accordance with some embodiments. The alpha power changes 900 reflect physiological effects of a stimulation 620 on neural tissues near the six electrodes. An alpha frequency is equal to 10 Hz. A stimulation 620 may be applied to one of the six bipolar electrode pairs. Across most channels, higher amplitudes and frequencies resulted in greater stimulation-related power increases.

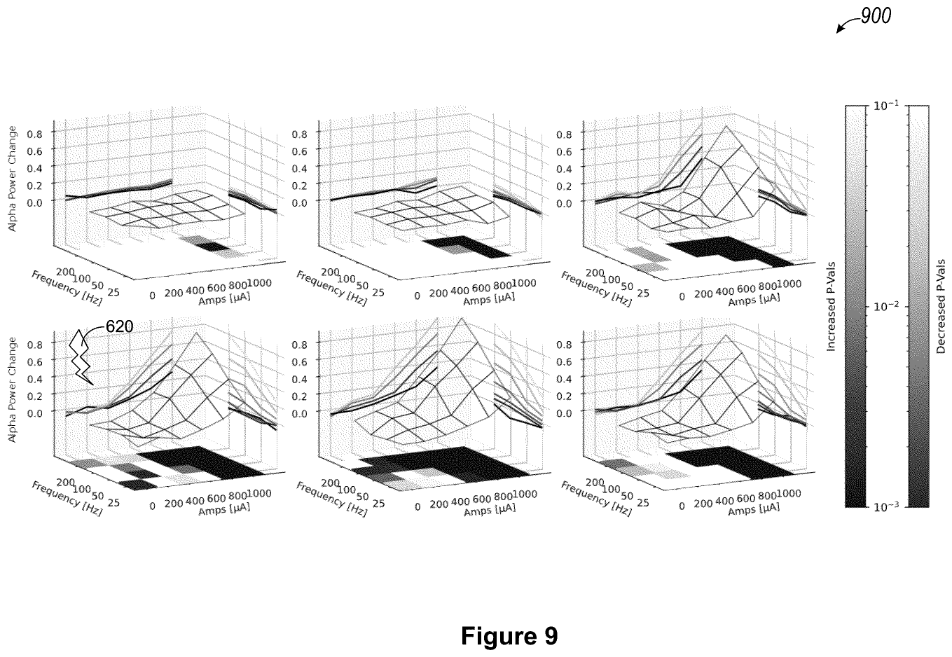

FIG. 10 shows a temporal diagram of a classifier output 1000, a correlation curve 1020 between a hit rate and a false alarm rate, a heatmap 1040 associated with bipolar electrode pairs, and temporal diagrams of two spectral powers 1060 and 1080, in accordance with some embodiments. A multivariate classifiers 116 includes a machine learning model (e.g., a logistic regression classifier) configured to predict periods 1002A and 1002B of movement and periods 1004 of stillness based on spectral features extracted from a plurality of electrodes 302 (e.g., forming a plurality of bipolar electrode pairs). For example, during a two-hour hold-out recording session, the classifier output 1000 indicates a probability of movement, tracking the periods 1002A and 1002B of movement and the periods 1004 of stillness. Ground truth movement or stillness states are determined based on accelerometer magnitude readings 1006 (e.g., sampled at 10 Hz and high-pass filtered with a cutoff at 0.1 Hz).

In some embodiments, the correlation curve 1020 between a fit rate and a false alarm rate represents a relation between true and false positives as a function of decision criterion, quantifying classifier performance in hold-out sessions. The correlation curve 1020 is plotted based on receiver operating characteristic (ROC) analysis, which is implemented to assess an accuracy of predictions by the multivariant classifier 116. An area under the ROC (AUC) is equal to 0.952±0.005. For comparison, a gray line 1022 shows a correlation for randomly permuted data.

In some embodiments, a Haufe method reveals the degree of influence of different features on classification performance while adjusting for the covariance structure in the model (e.g., multivariant classifier 116). The heatmap 1040 shows an increased weights on high frequencies and decreased weights on low frequencies, predicting a movement pattern corresponding to a variety of motor and cognitive operations.

In some embodiments, the temporal diagrams of two spectral powers 1060 and 1080 show a characteristic increase in low-frequency powers following a transition from locomotion to immobility. The two spectral powers 1060 and 1080 correspond to two adjacent electrode pairs that shares an electrode and the shaded interval 1090.

In some embodiments, it is determined whether signals recorded by the SNS could predict changes in the animal's behavioral state. To this end, data are recorded on the animal's activity level, gauged by a three-dimensional accelerometer attached to the animal's neck. Acceleration is recorded for each of the cardinal axes at 10 Hz during seven two-hour daytime sessions over two weeks. When the accelerometer sample magnitude exceeded a threshold value of 0.03 m/s², it is labeled “movement.” Otherwise, it is labeled as “stillness.” A criterion is further applied to assign labels to sustained periods of movement or stillness. Specifically, the number of “movement” samples are counted in a 10-second sliding window across all labeled samples and created a “high activity” event if the count exceeded 27. “Low activity” events are created by finding all spans of at least 600 “stillness” samples before the event and 150 “stillness” samples after the event. These values were selected to produce 150 to 250 events per typical 2-hour recording session. Applying this secondary criterion resulted in periods 1002A and 1002B of movement, periods 1004 of stillness, and non-labeled periods of ambiguous accelerometer readings.

In some embodiments, data are collected in the recorded sessions as input to a machine learning model (e.g., a logistic regression classifier) trained to discriminate brain activity predictive of whether the animal is moving or still. Spectral power is averaged across the time dimension for each “movement”/“stillness” event epoch (0-1000 ms relative to the event onset) as the input data. Thus, the features for each observed individual “movement” and/or “stillness” event were the average power across time, at each of the eight analyzed frequencies×N electrodes. In an example, an L2 penalization is applied to set a penalty parameter to 2.4×10⁻⁴ based on an optimal penalty parameter. The areas under the ROC curve 1020 (AUC) are computed to quantify classifier performance. The AUC measures a classifier's ability to identify true positives while minimizing false positives, In some embodiments, AUC is equal to 0.50.

In some embodiments, classifier performance is assessed using a leave-one-session-out (LOSO) method where, for each holdout session, a classifier is built based on the remaining sessions and used to predict event outcomes of the holdout session. The AUC was then calculated based on aggregated labeled and/or predicted outcomes for all holdout sessions.

Referring to FIG. 10, in some embodiments, panel A shows the results of cross-validation in an example two-hour session. Dots in periods 1002A and 1002B represent events labeled as “movement,” whereas dots in the period 1004 represent events labeled as “stillness.” These labels are derived using the motion detection algorithm described above. Periods that neither met our criteria for movement nor stillness have any dots.

In some embodiments, accelerometer readings 1006 suggest that during this two-hour interval, the animal exhibited three periods of significant movement activity and two periods of reliable stillness, as indicated by the corresponding dot clusters in periods 1002A, 1002B, and 1004. Panel A shows that dots in the period 1004 tend to fall under the 0.5 classifier output threshold, and dots in the periods 1002A and 1002B tend to stay above it, indicating that classifier predictions agreed with the accelerometer reading interpretations in most cases. To quantify model performance, the classifier's hit rate (classify high activity as high) and false alarm rate (classify low activity as high) are illustrated as a function of the criterion value. To the extent that the ROC curve 1020 rises above the positive diagonal line connecting (0, 0) and (1, 1) the model generalizes from the training to the test data, predicting periods of activity from spectral EEG features. To the extent that neural features reliably classify the animal's activity in holdout sessions, a high hit rate is found to correspond to a relatively low false alarm rate. Indeed, the curve rises quickly towards 1.0 as the false alarm rate increases, suggesting excellent classification of holdout data. The area under the ROC curve (AUC) quantifies classification performance. Averaging across seven holdout sessions, an average AUC value of 0.952±0.005 SEM is observed. The true ROC curves are compared to those created by applying the same classification model to data with permuted labels (shuffling labels across the events within each session). This “null” ROC curve appears as the curve 1022 in FIG. 10.

In some embodiments, previous analyses of neural data during motor tasks have identified a specific pattern of spectral power associated with movement. Specifically, during and immediately preceding movement, researchers have found increases in high-frequency power (>30 Hz) and decreases in low-frequency power. To determine whether a logistic regression classifier uncovered similar neural correlates of movement, the Haufe method is applied to the weights obtained from the optimal classifier fit to all seven sessions. This method transforms classifier weights to account for the covariances between features. Across most bipolar recordings, increased high-frequency power (squares with red shading) and decreased low-frequency power (squares with blue shading) mark high activity periods.

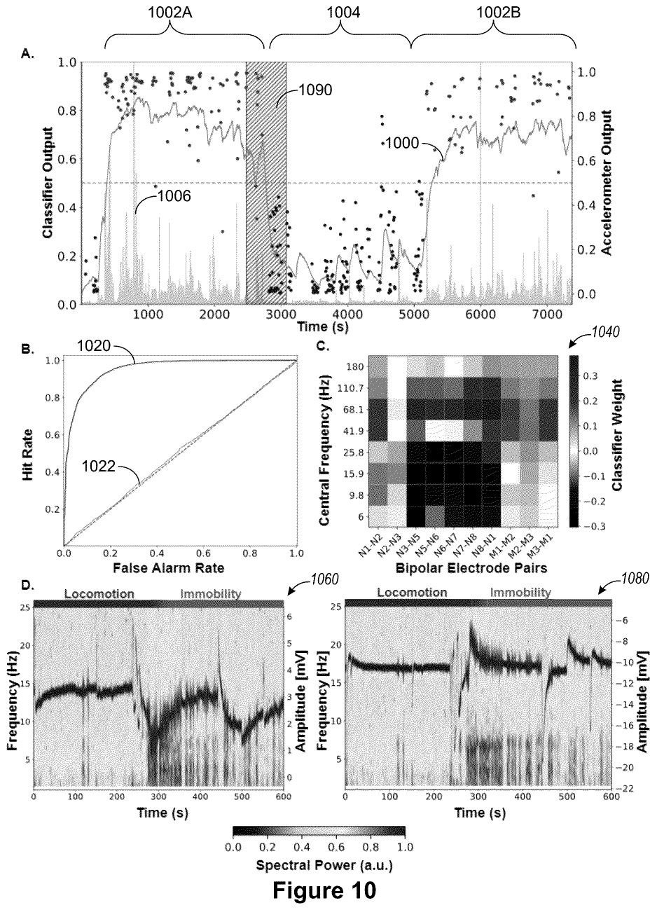

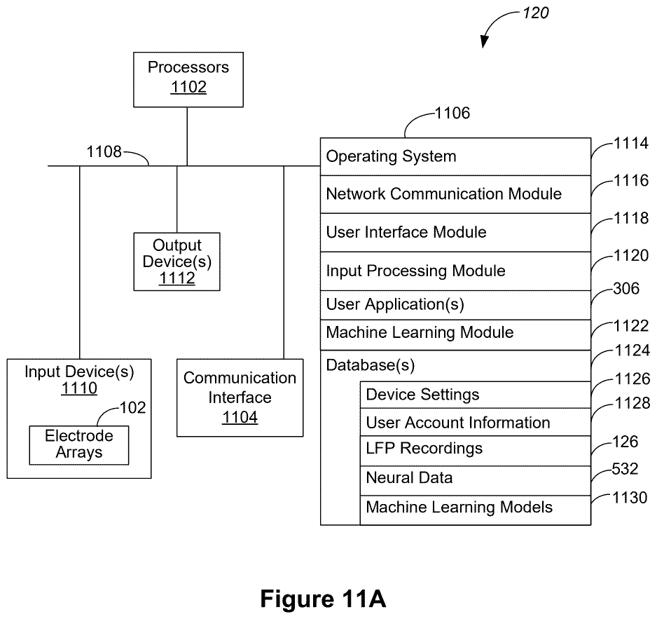

FIG. 11A is a block diagram of an example SNS 120, in accordance with some embodiments. The SNS 120 includes a plurality of electrodes 102, an implantable electronics package 104, and an external electronic system 106. The server 110 typically includes one or more processing units 1102, one or more communication interfaces 1104, memory 1106, and one or more communication buses 1108 for interconnecting these components (sometimes called a chipset). The one or more processing units 1102 include MCUs 508 and 514 and FPGA 509, 530, and 531. In some embodiments, the SNS 120 includes a user interface system that further includes one or more input devices 1110 (e.g., electrode arrays 102) or one or more output devices 1112.

Memory 1106 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. Memory 1106, optionally, includes one or more storage devices remotely located from one or more processing units 1102. Memory 1106, or alternatively the non-volatile memory within memory 1106, includes a non-transitory computer readable storage medium. In some embodiments, memory 1106, or the non-transitory computer readable storage medium of memory 1106, stores the following programs, modules, and data structures, or a subset or superset thereof:
Operating system 1114 including procedures for handling various basic system services and for performing hardware dependent tasks;
Network communication module 1116 for connecting each server 110 to other devices (e.g., server 110, computer device 108, or database 310) via one or more communication interfaces 1104 (wired or wireless) and one or more communication networks 314 (FIG. 1), such as the Internet, other wide area networks, local area networks, metropolitan area networks, and so on;
User interface module 1118 for enabling presentation of information (e.g., a graphical user interface for an application, widgets, websites and web pages thereof, and/or games, audio and/or video content, text, etc.) via one or more output devices 1112 (e.g., displays, speakers, etc.);
Input processing module 1120 for processing LFP recordings 126 that are measured by the electrode arrays 102;
User application 306 for execution by the SNS 120 to collect or process neural data 308 (e.g., including LFP recordings 126);
Machine learning module 1122 for applying machine learning models 1130 (e.g., multivariate classifiers 116, mnemonic prediction model 132); and
One or more databases 1124 for storing at least data including one or more of:
   Device settings 1126 including common device settings (e.g., service tier, device model, storage capacity, processing capabilities, communication capabilities, etc.) of the SNS 120;
   User account information 1128 for the user application 306, e.g., user names, security questions, account history data, user preferences, and predefined account settings of a user account of a particular patient 112 associated with the SNS 120;
   LFP recordings 126 and neural data 308 (e.g., neural data 532 in FIG. 5) provided by the SNS 120; and
   Machine learning models 1130 (e.g., multivariate classifiers 116, mnemonic prediction model 132), where in some embodiments, a first machine learning model is configured to determine a brain state (e.g., corresponding to a memory lapse event) based on LFP recordings 126, and in some embodiments, a second machine learning model is configured to determine stimulation parameters based on the brain state, neural data 308, and LFP recordings 126.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various embodiments. In some embodiments, memory 1106, optionally, stores a subset of the modules and data structures identified above. Furthermore, memory 1106, optionally, stores additional modules and data structures not described above.

FIG. 11B is a block diagram of a computer system 1150 that collaborates with the SNS 120 to provide a cloud-based neuromodulation platform, in accordance with some embodiments. The computer system 1150 includes a computer device 108, a server 110, or a combination thereof. The computer system 1150 typically includes one or more processing units (e.g., CPUs) 1152, one or more communication interfaces 1154, memory 1156, and one or more communication buses 1158 for interconnecting these components (sometimes called a chipset). The computer system 1150 includes one or more input devices 1160 that facilitate user input or one or more output devices 1162 including a display that enables presentation of user interfaces and display content.

Memory 1156 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. Memory 1156, optionally, includes one or more storage devices remotely located from one or more processors 1152. Memory 1156, or alternatively the non-volatile memory within memory 1156, includes a non-transitory computer readable storage medium. In some embodiments, memory 1156, or the non-transitory computer readable storage medium of memory 1156, stores the following programs, modules, and data structures, or a subset or superset thereof:
Operating system 1164 including procedures for handling various basic system services and for performing hardware dependent tasks;
Network communication module 1166 for connecting the computer device 108 or server 110 to other devices (e.g., server 110, computer device 108, the SNS 120, or database 310) via one or more communication interfaces 1154 (wired or wireless) and one or more communication networks 314 (FIG. 3);
User interface module 1168 for enabling presentation of information at each computer system 1150 via one or more output devices 1162 (e.g., displays, speakers, etc.);
Input processing module 1170 for detecting one or more user inputs or interactions from one of the one or more input devices 1160 and interpreting the detected input or interaction;
Web browser module 1172 for navigating, requesting (e.g., via HTTP), and displaying websites and web pages thereof;
User application 306 for execution by the computer system 1150 to collect or process neural data 308 (e.g., LFP recordings 126);
Machine learning module 1174 for training, deploy, or applying machine learning models 1182 (e.g., multivariate classifiers 116, mnemonic prediction model 132); and
One or more databases 1176 for storing at least data including one or more of:
   Device settings 1178 including common device settings (e.g., service tier, device model, storage capacity, processing capabilities, communication capabilities, etc.) of the computer system 1150;
   User account information 1180 for the user application 306, e.g., user names, security questions, account history data, user preferences, and predefined account settings of a plurality of user accounts associated with patients 112 or medical practitioners;
   LFP recordings 126 and neural data 308 (e.g., neural data 532 in FIG. 5) provided by the SNS 120 associated with each of the plurality of user accounts; and
   Machine learning models 1182 (e.g., multivariate classifiers 116, mnemonic prediction model 132), where in some embodiments, a first machine learning model is configured to determine a brain state (e.g., corresponding to a memory lapse event) based on LFP recordings 126, and in some embodiments, a second machine learning model is configured to determine stimulation parameters based on the brain state, neural data 308, and LFP recordings 126.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various embodiments. In some embodiments, memory 1156, optionally, stores a subset of the modules and data structures identified above. Furthermore, memory 1156, optionally, stores additional modules and data structures not described above.

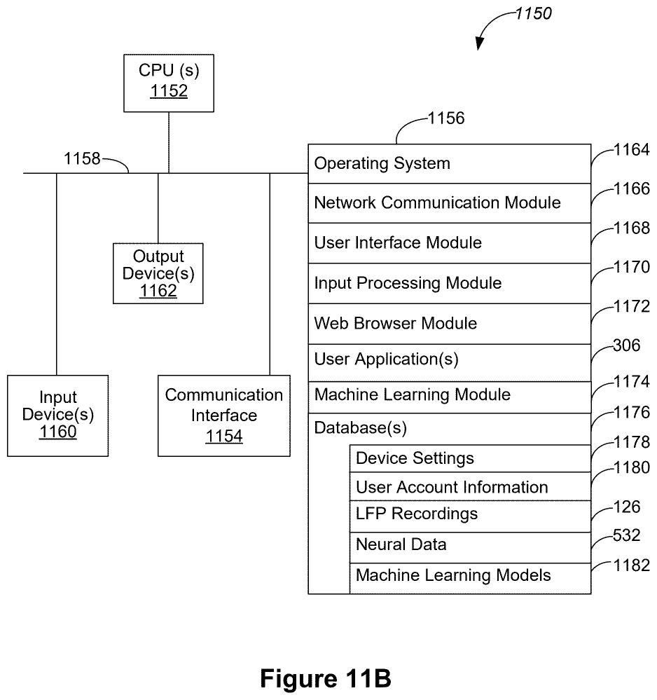

FIG. 12 is a flow diagram of an example method 1200 of implementing closed-loop neural stimulation for memory restoration, in accordance with some embodiments. Method 1200 is, optionally, governed by instructions that are stored in a non-transitory computer readable storage medium and that are executed (operation 1202) by one or more processors of an SNS 120. Each of the operations shown in FIG. 12 may correspond to instructions stored in the computer memory or computer readable storage medium (e.g., memory 1106 of the SNS 120 in FIG. 11A). The computer readable storage medium may include a magnetic or optical disk storage device, solid state storage devices such as Flash memory, or other non-volatile memory device or devices. The computer readable instructions stored on the computer readable storage medium may include one or more of: source code, assembly language code, object code, or other instruction format that is interpreted by one or more processors. Some operations in method 1200 may be combined and/or the order of some operations may be changed.

The method 1200 is implemented (operation 1202) at an SNS 120 including one or more electrode arrays 102, an implantable electronics package 104, and an external electronic system 106. The SNS 120 identifies (operation 1204) a plurality of channels 304 provided by a plurality of electrodes 302 of the one or more electrode arrays 102, and the one or more electrode arrays 102 are implanted in a brain region (e.g., the temporal lobe 410) of a patient 112. The SNS 120 collects (operation 1206) field potential (LFP) recordings 126 from the brain region of the patient 112 via the plurality of channels 304 provided by the one or more electrode arrays 102, and generates (operation 1208) a spectral decomposition of the LFP recordings 126 by the implantable electronics package 104. The implantable electronics package 104 is mounted on a skull under the scalp. The implantable electronics package 104 updates (operation 1210) a cognitive computational model 124 based on the spectral decomposition of the LFP recordings 126. Based on the cognitive computational model 124, the SNS 120 delivers (operation 1212) stimulation 620 to the brain region of the patient 112 via at least a subset of the one or more electrode arrays 102. The external electronic system 106 provides (operation 1214) power and data telemetry to the implantable electronics package 104 wirelessly.

In some embodiments, the external electronic system 106 is disposed in proximity to the implantable electronics package 104 and on top of the scalp, and the power and data telemetry are provided to the implantable electronics package 104 using a radio frequency (RF) coil system.

In some embodiments, the plurality of channels 304 includes 30, 32, or 64 channels.

In some embodiments, the implantable electronics package 104 further includes an electronics module that is hermetically packaged, one or more coaxial RF coils, and a subcortical probe (e.g., electrode arrays 102). The electronics module collects the LFP recordings 126 and generates the stimulation 620 without using a battery. The one or more coaxial RF coils receive power from, and exchange data with, the external electronic system 106. The subcortical probe is implanted in the brain region of the patient 112.

In some embodiments, the patient 112 is a human patient 112 with a traumatic brain injury (TBI).

In some embodiments, the implantable electronics package 104 further includes a communication module. The communication module obtains the cognitive computational model 124 from an external server 110.

In some embodiments, the external electronic system 106 receives the cognitive computational model 124 updated by the implantable electronics package 104 and generates a plurality of predefined stimulation patterns based on the cognitive computational model 124. The stimulation 620 is delivered based on the plurality of predefined stimulation patterns.

In some embodiments, the SNS 120 identifies at least the subset of the one or more electrode arrays 102 corresponding to one or more brain areas associated with memory performance (e.g., a memory lapse event determined in the model 124). The stimulation 620 is delivered to the one or more brain areas to activate the one or more brain areas.

In some embodiments, the implantable electronics package 104 processes the LFP recordings 126 to generate processed LFP recordings 126, and transmits the processed LFP recordings 126 to the external electronic system 106.

In some embodiments, the SNS 120 identifies at least the subset of the one or more electrode arrays 102 that are implanted within at least one of a temporal lobe, a frontal lobe, a middle temporal gyrus, a left parietal lobe, a hippocampus, an entorhinal cortex, and a right parietal lobe of the brain region.

In some embodiments, a computer system is communicatively coupled to the external electronic system 106. The computer system personalizes the cognitive computational model 124 for the patient 112 and provides the cognitive computational model 124 to the implantable electronics package 104 via the external electronic system 106.

In some embodiments, the LFP recordings 126 correspond to a moving sampling window, and the cognitive computational model 124 represents samples in the moving sampling window with a respective number of features for each of the plurality of channels 304. The respective number is smaller than a predefined feature limit.

Further, in some embodiments, the moving sampling window has a temporal length smaller than 800 millisecond. The predefined feature limit is equal to 8. A number of channels is less than or equal to 64. A footprint of the implantable electronics package 104 on the skull is less than 46 mm×37 mm. A power limit of the implantable electronics package 104 is 25 mW.

In some embodiments, the power provided to the implantable electronics package 104 is lower than a power limit, and is wirelessly transmitted over the scalp by an inductive link 204 coupled between the implantable electronics package 104 and the external electronic system 106.

In some embodiments, the one or more electrode arrays 102 includes four electrode arrays 102, and each electrode array further has sixteen electrodes 302 including a reference electrode.

In some embodiments, the subset of the one or more electrode arrays 102 includes two electrodes 302 on a first electrode array of the one or more electrode arrays 102. The implantable electronics package 104 generates biphasic, square-wave, stimulation pulses 534 and delivers the biphasic, square-wave, stimulation pulses 534 in a bipolar configuration to the brain region of the patient 112 via the two electrodes 302 on the first electrode array. Further, in some embodiments, based on the cognitive computational model 124, the SNS 120 determines a plurality of stimulation parameters including target electrode identifications, a pulse amplitude, a pulse width, and a stimulation duration.

In some embodiments (FIG. 1), the SNS 120 applies a multivariate classifier 116 to process the LFP recordings 126 or the spectral decomposition to update the cognitive computational model 124. The cognitive computational model 124 includes a distribution 118A or 118B of spectral power across electrodes 302, a subsequent recall status 122 of encoded items, and a mapping between the distribution 118A or 118B and the subsequent recall status 122.

In some embodiments, the cognitive computational model 124 124 includes a level of a mnemonic function 134. The SNS 120 applies a spectral filter 128 associated with a frequency range to the LFP recordings 126 126, estimates an LFP spectral power pattern 130 for each of the plurality of channels 304 at the frequency range, and applies a machine learning model (e.g., a mnemonic prediction model 132) to process the LFP spectral power pattern 130 and predict the level of the mnemonic function 134. The level of the mnemonic function 134 may indicate whether there is a memory lapse event. Upon detection of the memory lapse event, the SNS 120 may deliver the stimulation 620 to restore the memory for the patient 112.

In accordance with some embodiments of this application is at least a realization that direct electrical brain stimulation has emerged as a therapy for wide-ranging neurological and psychiatric disorders, including Parkinson's disease and essential tremor and more recently, intractable epilepsy, depression, and obsessive-compulsive disorder. With the exception of responsive stimulation for the treatment of epilepsy, other neurostimulation devices lack the ability to administer stimulation depending on brain state; more typically, stimulation occurs at regularly timed intervals independent of the state of the brain. Another class of emerging technologies aims to sense multi-channel brain activity for decoding purposes. Such devices may help treat patients with motor-neuron disease, spinal cord injury, or stroke, by detecting intended motor functions and controlling a neural prosthesis.

Some implementations of this application are directed to an SNS 120 configured to sense brain signals to detect momentary changes in brain function. Decoded signals determine when, where, and how to apply electrical stimulation 620, e.g., to facilitate memory restoration. In an example, the SNS 120 has 64 electrodes on four leads (e.g. electrode arrays 102), and is able to simultaneously measure local field potentials (LFP) across 60 bipolar contacts with reference to four electrodes, which serve as reference channels of four different leads, respectively. A closed loop system is formed based on the SNS's ability to both sense and stimulate the brain, and can be used to manage neurocognitive and affective disorders where functional impairments can vary dramatically from moment to moment and day to day. For example, stimulating the lateral temporal cortex during predicted memory lapses produces significant memory improvements in patients who had electrodes implanted for the treatment of pharmaco-resistant epilepsy. In another example, the SNS 120 is applied to enhance memory gains in a memory-impaired cohort of epileptic patients with a history of moderate-to-severe traumatic brain injury. The SNS 120 uses machine learning models (e.g., logistic regression classifiers) trained on spectral activity measured during learning, and may predict items that would be subsequently recalled or forgotten. In some situations, triggering stimulation on predicted memory lapses could acutely benefit memory. In some situations, mnemonic benefits only appeared for closed-loop stimulation (random stimulation did not improve memory).

In some embodiments, the SNS 120 is configured to deliver closed-loop stimulation chronically via an AI-enabled brain implant (e.g., the IPG 502), which communicates wirelessly with an external processor (EP) 504. The EP 504 is configured to be worn on the ear, similarly to a cochlear implant sound processor. The EP 504 also delivers power inductively to the IPG 502 via a rechargeable battery and acts as a communication hub between the IPG 502 and the cloud-based AI platform (e.g., including a computer device 108 and a server 110). Clinical programmer software, designed for future human studies, allows the SNS 120 to receive programming from patient-specific models of variable function. These models will trigger therapeutic stimulation, altering physiology based on variability in a particular patient's brain state. In the case of memory, predicted memory lapses will trigger stimulation, and classifier decoded memory output will allow for optimization of stimulation parameters.

In some embodiments, the SNS 120 records from a plurality of channels (bipolar electrode pairs) in a plurality of separate brain regions (e.g., for 60 channels in 4 brain regions). Memory-related brain signals occur across a distributed network of brain regions, requiring widespread surveillance capabilities of the SNS 120 and the ability to adaptively select among a larger set of stimulation targets. Large amount of LFP recordings 126 and neural data 308 can identify whole-brain patterns of neural activity that signal periods of good and poor memory. The basic capabilities of the SNS are demonstrated by a preclinical study in an ovine model. In some embodiments, two multi-contact depth leads are implanted: an 8-electrode lead designed specifically for the IPG and a commercially-available 4-electrode lead (e.g., Medtronic Model 3387S-40). Applying stimulation at a single pair of contacts on the SNS lead demonstrated that increasing stimulation amplitude and frequency systematically alters neural activity (alpha power) at the other recording electrodes. Further, in some embodiments, personalized machine learning models (e.g., logistic regression classifiers), trained on recording of neural data from all electrodes, reliably predict animal movement in hold-out sessions. These results demonstrate in vivo that the SNS 120 can detect and modulate behaviorally relevant neural signals.

In some embodiments, 60 channels are sufficient to reliably decode memory lapses in analyses of large open datasets. In some embodiments, the number of channels is greater than 60 and improves classifier performance and thereby improve parameter optimization and stimulation timing. In some embodiments, the electrode arrays 102 have higher spatial sampling. In some embodiments, the number of arrays is greater than 4.

Memory is also used to store instructions and data associated with the method 1200, and includes high-speed random access memory, such as DRAM, SRAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory, optionally, includes one or more storage devices remotely located from one or more processing units. Memory, or alternatively the non-volatile memory within memory, includes a non-transitory computer readable storage medium. In some embodiments, memory, or the non-transitory computer readable storage medium of memory, stores the programs, modules, and data structures, or a subset or superset for implementing method 1200.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various embodiments. In some embodiments, the memory, optionally, stores a subset of the modules and data structures identified above. Furthermore, the memory, optionally, stores additional modules and data structures not described above.
(A1) Some implementations of this application include a system for treating cognitive or neurological dysfunctions, comprising: one or more electrode arrays including a plurality of electrodes, the one or more electrode arrays configured to be implanted in a brain region of a patient and provide a plurality of channels based on the plurality of electrodes; an implantable electronics package coupled to the one or more electrode arrays, wherein the electronics package configured to be mounted on a skull under the scalp and including one or more processors and memory storing a cognitive computational model for memory enhancement and one or more programs for execution by the one or more processors, wherein the one or more programs comprise instructions for: concurrently collecting local field potential (LFP) recordings from the brain region of the patient via the plurality of channels provided by the one or more electrode arrays; generating a spectral decomposition of the LFP recordings; updating the cognitive computational model based on the spectral decomposition of the LFP recordings; and based on the cognitive computational model, delivering stimulation to the brain region of the patient via at least a subset of the one or more electrode arrays; and an external electronic system wearable by the patient, and configured to enable at least wireless data communication with the implantable electronics package.
(A2) In some embodiments of A1, the external electronic system is disposed in proximity to the implantable electronics package and on top of the scalp, and power and data telemetry are provided to the implantable electronics package using a radio frequency (RF) coil system.
(A3) In some embodiments of A1 or A2, the plurality of channels includes 30, 32, or 64 channels.
(A4) In some embodiments of any of A1-A3, the implantable electronics package further comprises: an electronics module that is hermetically packaged, wherein the electronics module is configured to collect the LFP recordings and generate the stimulation without using a battery; one or more coaxial RF coils configured to receive power from, and exchanging data with, the external electronic system; and a subcortical probe configured to be implanted in the brain region of the patient.
(A5) In some embodiments of any of A1-A4, the patient is a human patient with one of a traumatic brain injury (TBI), Alzheimer's disease, epilepsy, major depression, attention-deficit hyperactivity disorder (ADHD), Parkinson's disease, and essential tremor.
(A6) In some embodiments of any of A1-A5, the implantable electronics package further includes a communication module configured to obtain the cognitive computational model from an external server.
(A7) In some embodiments of any of A1-A6, the external electronic system is configured to receive the cognitive computational model updated by the implantable electronics package and generate a plurality of predefined stimulation patterns based on the cognitive computational model, and wherein the stimulation is delivered based on the plurality of predefined stimulation patterns.
(A8) In some embodiments of any of A1-A7, delivering stimulation further comprises identifying at least the subset of the one or more electrode arrays corresponding to one or more brain areas associated with memory performance, wherein the stimulation is delivered to the one or more brain areas to activate the one or more brain areas.
(A9) In some embodiments of any of A1-A8, the one or more programs stored on the memory of the implantable electronics package further comprise instructions for: processing the LFP recordings to generate processed LFP recordings; and transmitting the processed LFP recordings to the external electronic system.
(A10) In some embodiments of any of A1-A9, delivering stimulation further comprises identifying at least the subset of the one or more electrode arrays that are implanted within at least one of a temporal lobe, a frontal lobe, a middle temporal gyrus, a left parietal lobe, a hippocampus, an entorhinal cortex, and a right parietal lobe of the brain region.
(A11) In some embodiments of any of A1-A10, the system further includes a computer system communicatively coupled to the external electronic system, wherein the computer system is configured to personalize the cognitive computational model for the patient and provide the cognitive computational model to the implantable electronics package via the external electronic system.
(A12) In some embodiments of any of A1-A11, the LFP recordings correspond to a moving sampling window, and the cognitive computational model represents samples in the moving sampling window with a respective number of features for each of the plurality of channels, the respective number being smaller than a predefined feature limit.
(A13) In some embodiments of A12, the moving sampling window has a temporal length smaller than 800 millisecond; the predefined feature limit is equal to 8; a number of channels is less than or equal to 64; a footprint of the implantable electronics package on the skull is less than 46 mm×37 mm; and a power limit of the implantable electronics package is 25 mW or less.
(A14) In some embodiments of any of A1-A13, the external electronic system is configured to provide power to the implantable electronics package, and the power is lower than a power limit, and is wirelessly transmitted over the scalp by an inductive link coupled between the implantable electronics package and the external electronic system. The implantable electronics package includes a rechargeable configuration for storing the power.
(A15) In some embodiments of any of A1-A14, the one or more electrode arrays includes four electrode arrays, and each electrode array further has sixteen electrodes including a reference electrode.
(A16) In some embodiments of any of A1-A15, the subset of the one or more electrode arrays includes two electrodes on a first electrode array of the one or more electrode arrays, and delivering the stimulation further includes: generating biphasic, square-wave, stimulation pulses; and delivering the biphasic, square-wave, stimulation pulses in a bipolar configuration to the brain region of the patient via the two electrodes on the first electrode array.
(A17) In some embodiments of A16, delivering the stimulation further comprises: based on the cognitive computational model, determining a plurality of stimulation parameters including target electrode identifications, a pulse amplitude, a pulse width, and a stimulation duration.
(A18) In some embodiments of any of A1-A17, the one or more programs further comprising instructions for: applying a multivariate classifier to process the LFP recordings or the spectral decomposition to update the cognitive computational model, wherein the cognitive computational model includes a distribution of spectral power across electrodes, a subsequent recall status of encoded items, and a mapping between the distribution and the subsequent recall status, a spectral decomposition, a frequency-band power, and a plurality of target frequency ranges.
(A19) In some embodiments of any of A1-A18, the cognitive computational model includes a level of a mnemonic function, and the one or more programs further comprise instructions for one or more of: applying a spectral filter associated with a frequency range to the LFP recordings; estimating an LFP spectral power pattern for each of the plurality of channels at the frequency range; and applying a machine learning model to process the LFP spectral power patter and predict the level of the mnemonic function.
(A20) Some implementations of this application are directed to a method for treating cognitive or neurological dysfunctions, comprising: at a neuromodulation system including one or more electrode arrays, an implantable electronics package, and an external electronic system: identifying a plurality of channels provided by a plurality of electrodes of the one or more electrode arrays, the one or more electrode arrays being implanted in a brain region of a patient; collecting field potential (LFP) recordings from the brain region of the patient via the plurality of channels provided by the one or more electrode arrays; generating a spectral decomposition of the LFP recordings by the implantable electronics package, the implantable electronics package is mounted on a skull under the scalp; updating, by the implantable electronics package, a cognitive computational model based on the spectral decomposition of the LFP recordings; and based on the cognitive computational model, delivering stimulation to the brain region of the patient via at least a subset of the one or more electrode arrays; and enabling, by an external electronic system, wireless data communication with the implantable electronics package.
(A21) Some implementations of this application are directed to a neuromodulation system for treating cognitive or neurological dysfunction, comprising: a plurality of electrode arrays implantable in a brain region of the patient; a skull-mounted implantable electronics package coupled to the plurality of electrode arrays, the electronics package including one or more processors and memory storing one or more programs for execution by the one or more processors, wherein the one or more programs comprise instructions for: embedding a cognitive computational model; concurrently collecting local field potential (LFP) recordings from the patient via a plurality of channels located on the plurality of electrode arrays; updating the cognitive computational model based on the spectral decomposition of the LFP recordings; delivering stimulation to the patient in which the electronics package has been implanted, including stimulating, via at least a subset of the plurality of electrode arrays; an external electronic system wearable by the patient, and configured for enabling at least wireless data communication with the implantable package; and a cloud-based software system that personalizes the computational model for each patient.
(A22) In some embodiments of A21, power and data telemetry are provided to the implantable package using a radio frequency (RF) coil system.
(A23) In some embodiments of A21 or A22, the plurality of channels includes 64 channels.
(A24) In some embodiments of any of A21-A23, the skull-mounted implantable electronics package further comprises: a hermetically packaged battery-less electronics module for recording and stimulation; one or more coaxial RF coils for power and data telemetry; and a probe implanted in the brain region.
(A25) In some embodiments of any of A21-A24, the patient is a human patient with a traumatic brain injury (TBI), Alzheimer's disease, epilepsy, major depression, attention-deficit hyperactivity disorder (ADHD), Parkinson's disease, and essential tremor.
(A26) In some embodiments of any of A21-A25, the implantable electronics package obtains the cognitive computational model from an external server via a communications module in the electronics package.
(A27) In some embodiments of any of A21-A26, the external electronic system is configured to generate a plurality of predefined stimulation patterns based on the cognitive computational model; and wherein the stimulation is delivered based on the plurality of predefined stimulation patterns.
(A28) In some embodiments of any of A21-A27, delivering stimulation includes activating multiple brain areas associated with memory performance.
(A29) In some embodiments of any of A21-A28, the local field potential recordings are processed prior to transmission to an external electronic system.
(A30) In some embodiments of any of A21-A29, stimulation is delivered to at least one of a temporal lobe, a frontal lobe, a middle temporal gyrus, a left parietal lobe, a hippocampus, an entorhinal cortex, and a right parietal lobe of the brain region.
(A31) A non-transitory computer-readable storage medium, having instructions stored thereon, which are executed by one or more processors of a system in any of A1-A19 and A21-A30.

Some implementations of this application are directed to closed-loop neuromodulatory therapies, which are implemented with devices that can decode ongoing brain states and deliver multi-site stimulation. We describe the Smart Neurostimulation System (SNS), a cranially mounted implant with 60 configurable recording/stimulation channels, inductive power, and onboard spectral-feature classification. In three freely-moving sheep, we streamed local-field potentials and conducted two parameter-sweep experiments. Cross-validated movement classifiers achieved an average AUC exceeding 0.95. Increasing stimulation amplitude and frequency produced post-stimulation elevations in α-band (8-12 Hz) and γ-band (78-82 Hz) power at most target locations. The SNS unifies high-density sensing, real-time brain state decoding, and programmable closed-loop stimulation in a single device, demonstrating behavioral-state prediction and parameter-dependent neuromodulation in vivo. These findings establish a preclinical foundation for biomarker-guided stimulation targeting distributed cortical networks underlying memory and cognition.

In some embodiments, closed-loop neuromodulation—which adapts in real time to the brain's evolving state—offers a path to more effective and safer therapies across a wide range of neurological conditions. In epilepsy, responsive neurostimulation cuts seizure burden by stimulating the brain only when it senses preictal EEG patterns. In Parkinson's disease and essential tremor, adaptive deep-brain stimulation raises efficacy while reducing dyskinesias. In chronic pain and depression, state-contingent stimulation promises symptom relief without the habituation and mood swings seen with fixed schedules. These gains arise because closed-loop devices tailor pulse timing, location, and amplitude to moment-by-moment neural activity rather than relying on clinician-programmed settings that may be optimal only under the static conditions present at the time of programming.

In accordance with some embodiments of this application is at least a realization that adaptive systems rely on a strikingly small information footprint. An example adaptive system modulates amplitude from a single β-band power estimate per subthalamic lead, whereas the system for epilepsy responds to bandpass or line-length thresholds on up to four bipolar recording channels. Such low-dimensional control cannot capture the distributed, multifrequency dynamics that underlie higher-order functions such as memory, attention, or mood. Treating these complex indications, therefore, requires sampling many sites spanning distributed brain networks, extracting numerous spectral and temporal features simultaneously, and combining those features with a real time classifier capable of predicting momentary fluctuations in neurocognitive state. Prior work has shown that multivariate classifiers trained on hundreds of spectral features can reliably decode moments of high vs. low mnemonic efficacy in humans. In some embodiments, delivering such classifier-based closed-loop therapy, however, demands implants that record from many distributed sites, process spectral features onboard, and perform sensing and stimulation concurrently, all while remaining small, wireless, and power-efficient for chronic use.

Some implementations of this application are directed to a wireless, 60-channel, AI-enabled brain-computer interface, the Smart Neurostimulation System (SNS). Our proof-of-concept studies of closed-loop neuromodulation to improve memory motivated the SNS design. These studies, conducted in epilepsy patients with and without a prior history of traumatic brain injury (TBI), used external devices connected to a host PC to record brain activity and to apply stimulation through commercially available, FDA-cleared electrodes. Neurosurgeons implanted these electrodes semi-chronically to map seizure activity and brain function to guide potential resective surgery

In some embodiments, predicting momentary variability in human cognitive function motivated the requirements for the SNS. Mnemonic ability varies from trial to trial within the same individual, with the largest predictor being performance on the prior trial. Spectral analyses of intracranial recordings show that these fluctuations arise from large-scale network dynamics spanning lateral temporal, medial temporal, and prefrontal cortices. Reliable prediction of memory success requires simultaneous sampling from dozens of electrodes and the use of multivariate models that integrate information across frequencies and regions. These findings highlight the need for a neural interface capable of wide-area sensing, real-time spectral estimation, and rapid classifier-based control of stimulation.

In some situations, mnemonic effects of electrical stimulation depend on brain state: stimulation impaired memory when delivered during classifier-predicted good states and improved memory when delivered during classifier-predicted poor states. In a subsequent study, we validated these observations by designing a closed-loop system that triggered high-frequency (100-200 Hz) stimulation bursts upon detected memory lapses, reliably improving memory for stimulated items. A replication in a cohort (N=8) of epilepsy patients with moderate-to-severe TBI further showed that the mnemonic benefits of closed-loop stimulation accrue to the entire stimulated list, not just to the stimulated items. Analyses of a larger cohort of patients (N=47) found that only stimulation near white-matter tracts yielded consistent list-level mnemonic benefits; among these targets, those with the strongest functional connectivity to the memory network produced greater mnemonic boosts. These studies provide proof of concept for a brain-computer interface therapy for memory loss. Approved devices, however, cannot meet the multichannel sensing and closed-loop stimulation requirements of such a therapy.

### 1. The Smart Neurostimulation System (SNS)

FIG. 1 is a flow diagram illustrating an example process 100 of applying a smart neurostimulation system (SNS) 120, in accordance with some embodiments. The SNS includes (a) a cranial implant responsible for neural sensing and stimulation; (b) a wearable External Processor providing power and acting as a communications hub; (c) four depth leads with a total of 64 contacts; and (d) a cloud-based AI platform that connects to the SNS via a Clinical Programmer device or Mobile App. Clinicians optimize the therapy for each patient using the programmer. Patients perform delayed recall tasks (e) while the SNS analyzes spectral features (f) learning to predict, in hold-out sessions, which list items subjects will remember (red dots) or forget (blue dots) (g). The SNS uses these models to trigger stimulation during predicted memory lapses and to optimize stimulation parameters. The SNS comprises a cranially mounted implantable pulse generator (IPG) for processing neural data and controlling therapy delivery; a wearable external processor (EP) that powers the IPG and acts as a communication hub; four custom depth leads for sensing and stimulating neural tissue; and a physician programmer and cloud-based AI platform developed for personalizing therapy in human studies. During therapy personalization, the SNS records multi-channel field potentials while patients study and recall word lists). Standardized delayed-recall tasks serve as validated surrogates for everyday memory, with performance predicting functional outcomes, such as return to work, in TBI cohorts.

In some embodiments, applying spectral filters to the EEG data enables the IPG to estimate LFP power at frequencies from 3-180 Hz. The spectral filters employ a standard Goertzel algorithm for estimating signal power at a specific frequency. The firmware specifies the filter coefficients, which can be easily updated. Machine-learning models trained on these spectral features predict mnemonic success and drive later closed-loop stimulation decisions during therapy.

FIG. 2B is a diagram of an example smart neurostimulation system prototype, including a) the implantable pulse generator; b) the external processor; and c) depth leads containing up to 16 electrodes each. All device materials were selected for biocompatibility and prior medical device usage. The silicone-encapsulated IPG houses a hermetically sealed titanium can surrounding the embedded electronics, platinum/iridium, stainless steel and pellethane in the lead connector assembly, and Parylene-C surrounding the gold-coated magnet. The depth lead utilizes platinum/iridium electrodes, a stainless-steel set screw block, and a pellethane lead body. EP materials include ABS plastic, nylon and silicone. A U.S. quarter illustrates the scale of the device. The IPG senses local field activity from 60 electrodes (plus four fixed reference electrodes), processes the data and controls therapy delivery. The EP inductively powers the IPG and relays data over a 2.2 Mbit/s link, sufficient to stream (500-Hz sampled) signals from every channel, with reliable transmission across 5-10 mm of scalp.

In some embodiments, the EP serves as the system's communication hub, providing USB for high-speed communication to the Programmer and Bluetooth low energy (BLE) for low-bandwidth mobile-app telemetry. The SNS end-to-end authenticates and encrypts all commands and responses using 128-bit AESGCM, ensuring only authenticated users and devices can connect to the device, and preventing administration of malicious therapy parameters. As with cochlear implants, users recharge the EP overnight, outside therapy hours. The preclinical version of the EP demonstrated here has 256 Mbits of on-board FLASH storage, capable of storing 30 minutes of spectrally transformed neural data from all 60 sensing channels. The clinical release of the EP will include 2 Gbits of storage, capable of storing more than 4 hours of spectrally-transformed neural data from all 60 sensing channels. During clinician programming over a USB connection, the programmer laptop operates on battery power, eliminating any connection between the EP and the power mains. During routine at-home use, the EP transfers data to the Cloud each night via a BLE connection to the user's mobile phone.

In some embodiments, the SNS design arose from a need to meet three constraints: (i) recording field potentials from ~64 electrodes in widespread brain locations at sampling rates ≥500 Hz (to discern high-frequency signals that predict mnemonic function); (ii) delivering rapid stimulation bursts triggered by onboard machine-learning models that predict memory lapses; and (iii) fitting within a cranial form factor proven safe.

To estimate the minimum number of electrodes required to achieve classification performance comparable to that reported in our prior studies, we conducted a feature subset resampling analysis using publicly available intracranial EEG data from the Restoring Active Memory project. We identified participants with at least 128 recording channels and trained penalized logistic regression classifiers to predict mnemonic success from spectral features of EEG activity measured during word encoding. For each iteration, we randomly sampled subsets of 4, 8, 16, 32, or 64 electrodes and computed the cross-validated area under the receiver operating characteristic curve (AUC). Relative to classifiers trained on the full feature set, we observed statistically significant reductions in AUC for models trained on subsets of 4, 8, 16, or 32 electrodes. In contrast, performance for models trained on 64 electrodes did not differ significantly from that obtained using all channels (both mean AUC ~0.62, evaluated on held-out sessions). These findings indicate that approximately 64 channels are sufficient to reproduce the decoding performance achieved in our closed-loop stimulation studies, thereby supporting the feasibility of memory-state classification with the SNS.

FIG. 5 is a schematic diagram of an example SNS system hardware and electrical interface, in accordance with some embodiments. The SNS system includes a) the External Processor (EP), and b) the Implantable Pulse Generator (IPG). c) Expanded view of the electrical interface between the stimulation/sensing ASIC (Neuromodulation Integrated Circuit; NMIC) and the leads. d) the Electrode-Tissue interface R-C model. Information (data or commands) can be sent via the EP (a) to the IPG (b) and in reverse. The IPG records neural data via (c) and transmits it to the EP (which can then communicate with an external program (PC). The electrical interface and Electrode-Tissue interface (c) and (d) illustrate both sensing (1) and stimulation (2) circuit models.

In some embodiments, the EP and IPG communicate via an inductive link composed of two conductive coils (FIG. 5). Command and response data flow bidirectionally through this link, while the IPG simultaneously transmits neural data to the EP. The EP can then send neural data to a PC over USB or to a mobile phone via BLE. A microcontroller (MCU) in the EP controls most of the system logic, including communication, safety systems, and firmware updates. A field programmable gate array (FPGA) controls the inductive link communication protocol on both the EP and the IPG. Power management hardware on the IPG regulates power input, while MCUs on the IPG and EP process all communication messages and manage therapy safety.

In some embodiments, the electrical interface is formed between IPG and neural tissue. The IPG lead interface holds four leads with 16 electrodes each. Sixty of the 64 platinum-iridium electrodes can sense and stimulate neural tissue; a central electrode on each lead serves as an electrical reference. On the IPG, a proprietary Application-Specific Integrated Circuit (ASIC) connects to the electrode contacts through DC-blocking capacitors. This custom ASIC was designed specifically for brain interfaces to provide sensing and stimulation capabilities. Inside the ASIC, there are 64 channels for sensing and stimulation, one for each electrode contact. The ASIC has four independent current sources that can multiplexed to any of the 64 channels, delivering square-wave, charge-balanced, biphasic pulses. At the system level, our software for the forthcoming clinical trial limits stimulation to two simultaneous bipolar targets. For our clinical application, neural data is only analyzed during the post-stimulation periods to avoid contamination with stimulation artifact.

FIG. 6 is a flow diagram of an example neural modulation process 600 implemented to record neural activities and deliver neural stimulations, in accordance with some embodiments. The SNS includes firmware running on both the IPG (a) and the External Processor (b) to sense neural data and administer electrical stimulation. Once it is sensed from the brain, neural data can take two paths (numbered): 1.) a Streaming path where data is sent to an external computer via USB; and 2.) a Therapy Delivery path where the data are processed and therapy is administered based on brain state classification. Brain state classification and therapy delivery blocks are programmable and adaptable (marked with a gear icon).

In some embodiments, the SNS firmware architecture (FIG. 6) supports command and control, battery management, firmware updates, event logging, and over-the-air updates. The firmware operates in two modes: (i) Therapy Personalization and (ii) Therapy Delivery. Therapy Personalization mode supports the transmission of raw 500-Hz LFP data from the IPG to an external PC (via the EP) for offline processing and storage (see Streaming Path FIG. 6). Therapy Delivery mode supports spectral processing, brain state classification and stimulation delivery (see Therapy Delivery path in FIG. 6). In this mode, the IPG estimates spectral power and makes therapy decisions every 128 ms. If the classifier indicates that therapy should be applied, the IPG applies electrical stimulation to the brain within 25 ms of the last neural data sample entering the buffer. Therapy parameters, including target electrodes, stimulation amplitude (0.02-5.0 mA), frequency (15-256 Hz), pulse width (16-500 μsec), and duration (1-2000 msec) are configurable by the user. Therapy Delivery mode also supports the streaming of processed (spectrally transformed) neural data from the IPG to the EP for offline processing and storage. In some embodiments, the IPG utilizes cyclic on-off keying (COOK) for high-speed data transmission, passively modulates the inductive field for efficient communication, and has been designed to operate continuously while staying within safe thermal limits as set by ISO 14708-1.

In an example, the IPG consumes 20 mW when running autonomously in Therapy Delivery mode (and slightly more when sending data to the EP). Taking into account the ~50% power delivery efficiency of the inductive link, the total power consumption of the system is 40 mW, within the range of commercially available cochlear implants (e.g., 20 mW to 100 mW). The EP can support more than 8-hours of use per charge with a 210 mAh Lithium-ion battery, and we expect battery life to significantly increase over time with improved battery technologies and system efficiencies. For our forthcoming clinical study, we plan to provide patients with a second EP device to support 16 hours per day of therapy delivery.

### 2. Preclinical Ovine Study

To evaluate the performance of the Smart Neurostimulation System (SNS), we conducted a preclinical study using an ovine model. FIG. 7A is a flow diagram of an example preclinical study process 700, in accordance with some embodiments, and FIG. 7B illustrate an example surgical procedure 750 for chronic animal testing, in accordance with some embodiments. Time course of the preclinical study. CT imaging was collected on Day −7, Day 0 (post-implantation) and Day 120 (prior to study exit). (B) The IPG and a protective exoskeleton were implanted into a pocket in the neck, and two leads were implanted into the left and right parietal lobes. A preclinical EP device bandaged on the skin overlying the IPG coil (not shown) communicated with and powered the IPG. (C, D, E) CT imaging showing the locations of the depth leads in animals S001, S002 and S003, respectively.

In some embodiments, CT imaging is performed to guide trajectory planning for electrode implantation. On Day 0, a functional neurosurgeon (R.E.G. or B.C.L.) implanted a single SNS IPG and two depth leads per animal, followed by post-operative CT scans to confirm lead placement. Each animal was allowed a six-day recovery period before initiating neural recordings and stimulation.

In some embodiments, the IPG was designed for installation into the human cranium. However, anatomical constraints prevent installation in the sheep cranium. Therefore, the IPG was installed in the neck of the animal, housed in an exoskeleton that protected the IPG coil from large flexion forces during movement, which could damage the IPG.

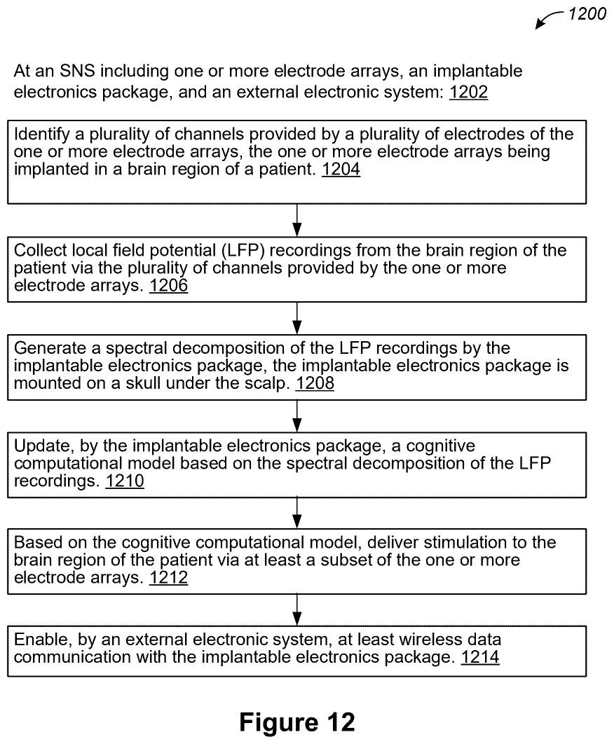

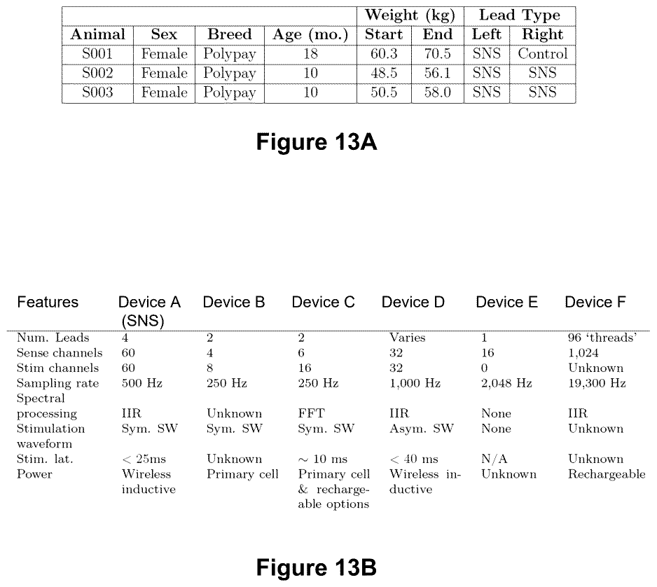

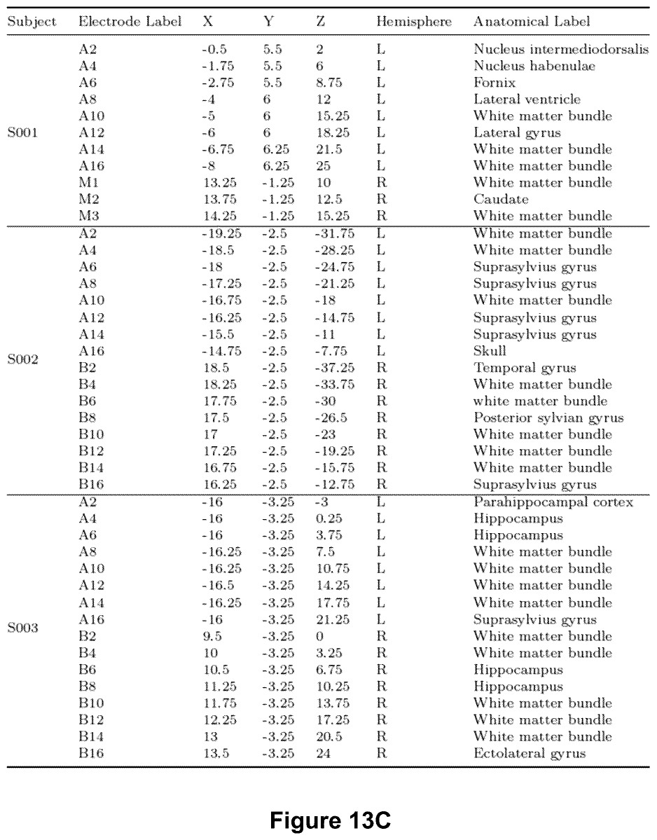

Under some circumstances, animals were housed under veterinary supervision with free movement within their enclosures throughout the study. At the study's conclusion, each animal was humanely euthanized. FIG. 13A shows animal characteristics and depth leads implanted into left and right hemispheres, in accordance with some embodiments.

In some embodiments, to localize the electrodes anatomically, we registered post-operative CT scans to the sheep brain MRI atlas. The iterative metal artifact reduction (iMAR) algorithm was applied to reduce CT signal distortion from the electrodes. We used bright metal artifacts to identify electrode positions and extracted 3D coordinates relative to anatomical regions defined by the atlas. FIG. 13C reports the anatomical locations for all monopolar electrodes in some embodiments.

In some embodiments, each SNS lead included eight channels, numbered with even integers from 2 to 16. Channel 8 served as the sensing reference, and all reference electrodes were internally connected within the IPG. (e.g., where in some situations, a control lead does not include a reference electrode and thus not contribute to the reference scheme in animal 1.) For analysis, we generated bipolar “virtual” recordings by subtracting voltages between adjacent non-reference electrodes (e.g., A2-A4). Bipolar referencing, computed as the voltage difference between pairs of adjacent electrodes, filters out signals common to both channels, thereby improving signal-to-noise ratio. This referencing scheme attenuates oculomotor and electromyographic artifacts that can mix with neural signals, and large-N human studies have shown that bipolar referencing of intraparenchymal electrodes better resolves the neural correlates of memory than average referencing. On each SNS lead, the fourth most distal electrode (denoted number eight) served as the electrical reference for the other electrodes. We developed a small computing system (e.g., based on a Raspberry Pi processor) to collect data from the preclinical EP and store it on SD cards for subsequent offline analyses, described below.

In some embodiments, three animals underwent surgical implantation without intraoperative complications. The most commonly observed adverse effect was localized swelling at the IPG site, which resolved with pressure bandaging. Routine veterinary assessments confirmed normal wound healing, and there was no evidence of infection, hemorrhage, or tissue damage at the lead implantation sites. One serious adverse event occurred in Animal 3: erosion of the skin overlying the IPG coil. This was attributed to an EP redesign intended to improve its physical robustness that reduced the coil surface area and increased the coil magnetic force, unintentionally increasing the skin pressure beyond the safe limit of 3.7 kPa. We have since decreased the EP magnet strength to keep skin pressure below the safe limit.

### 3. Using Neural Recordings to Predict Movement

We first asked whether the SNS could record and decode neural signals related to animal behavior. With a triaxial accelerometer sampling at 10 Hz, we gauged the animal's activity level throughout two-hour sessions. We selected non-overlapping 1-second epochs from longer sustained periods of movement or stillness, and used the neural data from these epochs to classify movement in hold-out sessions. Whenever an accelerometer reading exceeded 0.03 m/s², and more than 30% of samples in the following 10 seconds exceeded the accelerometer threshold, we created a “movement” epoch. Whenever an accelerometer reading was below 0.03 m/s², if all samples in the prior 60 and subsequent 15 seconds were below the threshold, we created a “stillness” epoch. We included sessions that had at least 100 movement and 100 stillness epochs, and neither class made up more than 80% of the total epochs.

In some embodiments, all animals had one SNS IPG implanted at the bottom of the neck and one depth lead placed into each hemisphere. In animal S001, one 8-channel SNS depth lead was placed in left hemisphere and a control lead was placed in right hemisphere. All other animals had SNS 8-channel depth leads placed into each hemisphere.

In some embodiments, the final dataset comprised 48, 36, and 48 sessions from the three sheep, respectively. For each one-second epoch, we calculated the spectral powers using Morlet wavelets at eight log-spaced frequencies, ranging from 6 to 180 Hz. The powers were then log-transformed and z-scored within each recording channel and frequency, and then averaged across the one-second epoch. The spectral powers for every epoch at each frequency and channel served as the features input to a machine learning model (e.g., an logistic regression classifier), where the labels indicated the movement or stillness identity of each epoch. Using an 80-20 session split, we trained the classifiers to discriminate brain activity predictive of movement and stillness. For cross-validation, we randomly repeated the 80-20 session split, with the number of permutations equal to the number of sessions. We computed the area under the receiver operating characteristic curve (AUC) to quantify classifier performance for each permutation.

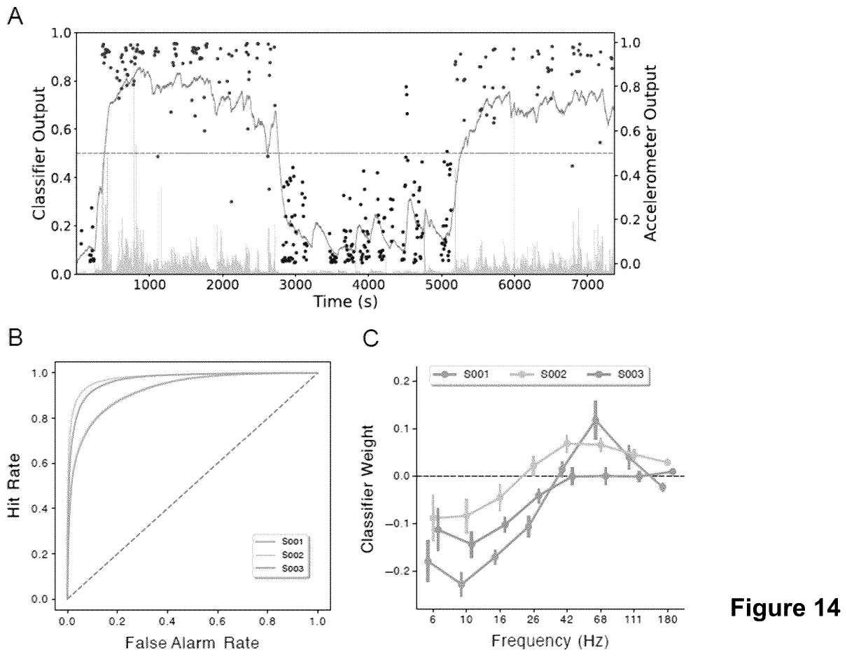

FIG. 14 is a set of diagrams for classifying animal movement, in some embodiments. For a first figure (A) of FIG. 14, we trained a machine learning model (e.g., an L2 logistic regression classifier) to predict periods of movement vs. stillness based on spectral features. Across a two-hour hold-out session, we show that classifier output probability (gray) tracks periods of movement (red) and stillness (blue). These representative periods were chosen based on the accelerometer magnitude readings (cyan) sampled at 10 Hz and high-pass filtered with a cutoff at 0.1 Hz. For a second figure (B) of FIG. 14, a ROC analysis, which shows the relation between true and false positives as a function of decision criterion, quantifies classifier performance. For a third figure (C) of FIG. 14, The Haufe method reveals the degree of influence of different features on classification performance while adjusting for the covariance structure in the model. Here we see that increased weights on high frequencies and decreased weights on low frequencies predicted movement. Error bars represent one standard error of the mean across recording channels.

The first figure (A) of FIG. 14 demonstrates the cross-validation in an example session. Accelerometer readings, in cyan, suggest the animal exhibited two periods of reliable stillness and two periods of significant activity. Red and blue dots indicate classifier outputs for epochs labeled as “movement” and “stillness”, respectively. Blue dots tend to fall under the 0.5 classifier output threshold, and red dots tend to stay above it, indicating that classifier predictions agreed with the accelerometer reading interpretations in most cases.

We illustrate receiver operating characteristic (ROC) curves in the second figures (B) of FIG. 14. For each animal, the hit rate rises quickly towards 1.0 as the false alarm rate increases, suggesting neural features reliably classify the animal's activity in holdout data. Across the cross-validation test partitions, we found AUC values of (M=0.924, SD=0.031), (M=0.979, SD=0.008), and (M=0.969, SD=0.008) for the three sheep. As a control analysis, we repeated the cross-validation with shuffled movement/stillness labels for the training and test data, finding AUC values of (M=0.500, SD=0.005), (M=0.502, SD=0.006), and (M=0.499, SD=0.005). We also trained movement classifiers for each animal on data from the first half of the study and tested these classifiers on data from each week within the second half of the study. We found that classifiers generalized very well from the first half to the second half of the study, with average AUCs of 0.929 (SD=0.013), 0.962 (SD=0.038), and 0.973 (SD=0.011), for the three animals. These findings demonstrate the stability of the SNS' neural recordings and classifier weights across many weeks of recordings.

Previous analyses of neural data during motor tasks have identified increases in high-frequency power (>30 Hz) and decreases in low-frequency power during and immediately preceding movement. To determine whether our classifier uncovered similar neural correlates of movement, we applied the Haufe method to the weights obtained from the optimal classifier fit to cross-validation training datasets. This method transforms classifier weights to account for the covariances between features. In the third figure (C) of FIG. 14, we illustrate that increased high-frequency power and decreased low-frequency power tended to mark high activity periods. Although aggregating across all electrodes illustrated a similar trend across animals, the classifiers identified the unique pattern of neural activity related to behavior within each animal.

### 4. Neural Recordings and Stimulation Performance

In some embodiments, to evaluate the SNS's ability to modulate neural activity, we ran two stimulation experiments. We compared spectral power in the 200-950 ms post-stimulation period to power in the −750-0 ms pre-stimulation period. Analyzing the post-stimulation period, isolated stimulation induced brain activity that persists beyond the stimulation interval, and the 200-ms poststimulation buffer attenuated potential stimulation artifact. Guided by prior work on spectral biomarkers of cognition, we focused on alpha (8-12 Hz) and gamma (78-82 Hz) band power. We used Welch's method to calculate the power spectral densities during the pre- and post-stimulation periods, which does not require a buffer that risks leakage from the stimulation period. Before further analysis, we log-transformed each power value, as in previous work, and calculated the z-scores of spectral powers within each frequency, channel, and session. For every stimulation event, i, within each frequency, f, and channel, c, we computed the difference in post- and pre-stimulation power as Δ_{P(f,c,i)}=P(f, c, i)post−P(f, c, i)pre (e.g., in the third figure (C) of FIG. 14).

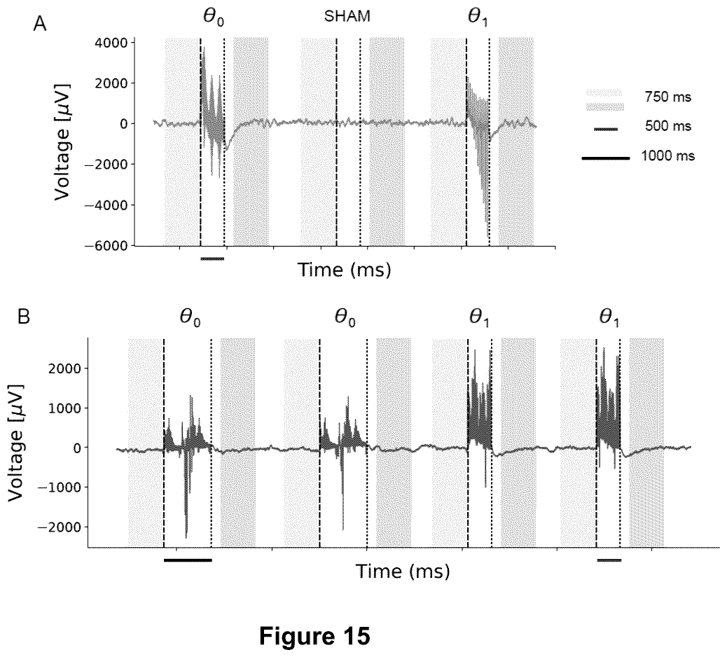

FIG. 15 is a set of diagram showing results of parameter search, in accordance with some embodiments. We applied stimulation with varying parameter sets θ and analyzed spectral powers from −750 to 0 ms (light gray) before stimulation onset (dashed line) and 200 to 950 ms (dark gray) following stimulation offset (dotted line). In Experiment 1, the SNS delivered 500 ms bi-phasic stimulation bursts at varying amplitudes [Sham, 0.2, 0.4, 0.6, 0.8, 1 mA] and frequencies [f=25, 50, 100 and 200 Hz] at a single target location (A10-A12), with the parameter set updating each stimulation event. Example field potential recorded from a bipolar electrode pair (A14A16). (B) In Experiment 2, the SNS delivered bi-phasic stimulation bursts at varying amplitudes [0.5, 1, 1.5 mA], frequencies [100, 200 Hz], and durations [500, 1000 ms] at twelve target locations, with the parameter set updating every two stimulation events. Example field potential recorded from a bipolar electrode pair (B4-B6).

Experiment 1, conducted on S001, tested whether the SNS could reliably modulate neural activity. Applying stimulation at varying amplitudes and frequencies at a single pair of neighboring electrodes, we evaluated power changes at the remaining non-anode, non-cathode electrodes on the same implanted lead. On each trial, the SNS applied 300 μs biphasic stimulation pulses at frequencies of 25, 50, 100 and 200 Hz and amplitudes of 0 (sham), 0.2, 0.4, 0.6, 0.8, and 1 mA across a bipolar pair of neighboring electrodes (A10-A12) in left frontal white matter. To minimize order effects, we shuffled stimulation trials to create 10 uniquely ordered session protocols, each with 30 trials of each of the 24 frequency×amplitude combinations. Each trial applied stimulation for 500 ms, followed by a 2.0-2.25 s inter-trial interval (FIG. 15). One sheep completed 124 sessions.

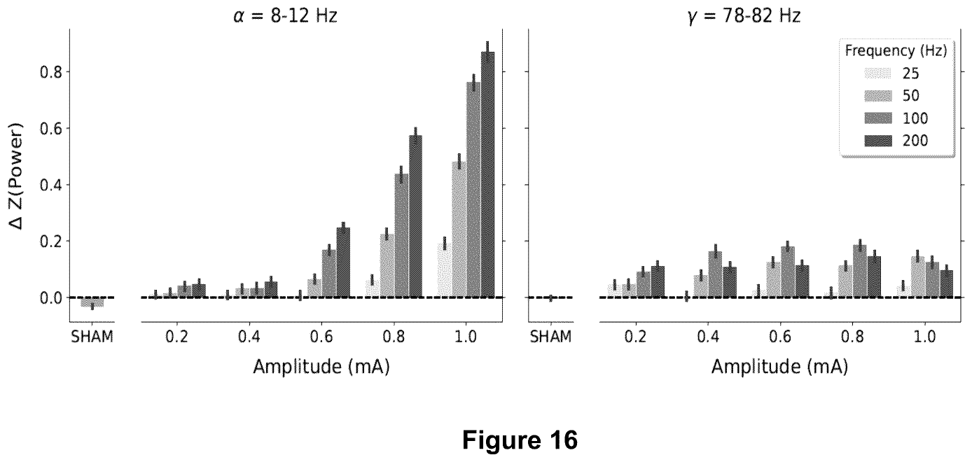

FIG. 16 is a set of diagrams showing stimulation's physiological effects, in some embodiments. The effect of stimulation amplitude and frequency on alpha-band power (8-12 Hz, left panel) and gamma-band power (78-82 Hz, right panel). This analysis averages over the three electrode pairs that do not overlap with either the anode or cathode. Increasing stimulation amplitude and frequency results in greater power increases.

Increasing stimulation amplitude and frequency boosted both alpha and gamma power (FIG. 16). These effects broadly align with prior stimulation parameter search experiments in humans. We evaluated these trends with a linear mixed effects model containing fixed effects for stimulation amplitude, frequency, and their interaction, a random intercept for session, and had predictor variables centered and normalized to the range 1 to 1. Our model identified statistically significant linear effects of both stimulation amplitude β=0.311, CI=[0.303, 0.319] and frequency, β=0.140, CI=[0.134, 0.147] on stimulation-related changes in alpha power. Our model also identified a significant interaction between amplitude and frequency, β=0.159, CI=[0.149, 0.168], as seen in the increasing strength of the amplitude-power relation for increasing stimulation frequency evident in FIG. 15. We observe significant relations of stimulation amplitude, β=0.013, CI=[0.005, 0.022], frequency, β=0.032, CI=[0.025, 0.040], and their interaction, β=−0.011, CI=[−0.022, −0.001], with changes in gamma power. These results align with previous work finding post-stimulation increases in high-frequency activity (HFA). Prior research has shown that HFA increases correlate with increases in neural firing rate, linking stimulation-related spectral changes to the modulation of neural activity.

In some embodiments, experiment 2 further explored the modulatory capabilities of the SNS. In two animals (S002 and S003), we stimulated 12 neighboring electrode pairs with varying amplitude, frequency, and duration. We evaluated stimulation's effects on power at the remaining electrodes on the same implanted lead not involved in stimulation and the electrodes on the other implanted lead. On each trial, the SNS applied 300 μs biphasic stimulation pulses at frequencies of 100 and 200 Hz, amplitudes of 0.5, 1, and 1.5 mA, and durations of 500 and 1000 ms. As illustrated in FIG. 15, stimulation trials occurred in blocks of two stimulation events with the same stimulation parameters. Within a block, the two stimulation events occurred with a random inter-trial interval of either 1000-1250 ms or 2000-2250 ms. Across blocks, there was a random 2000-2250 ms inter-stimulus interval. We collected a total of 42 and 154 sessions from the two sheep, respectively.

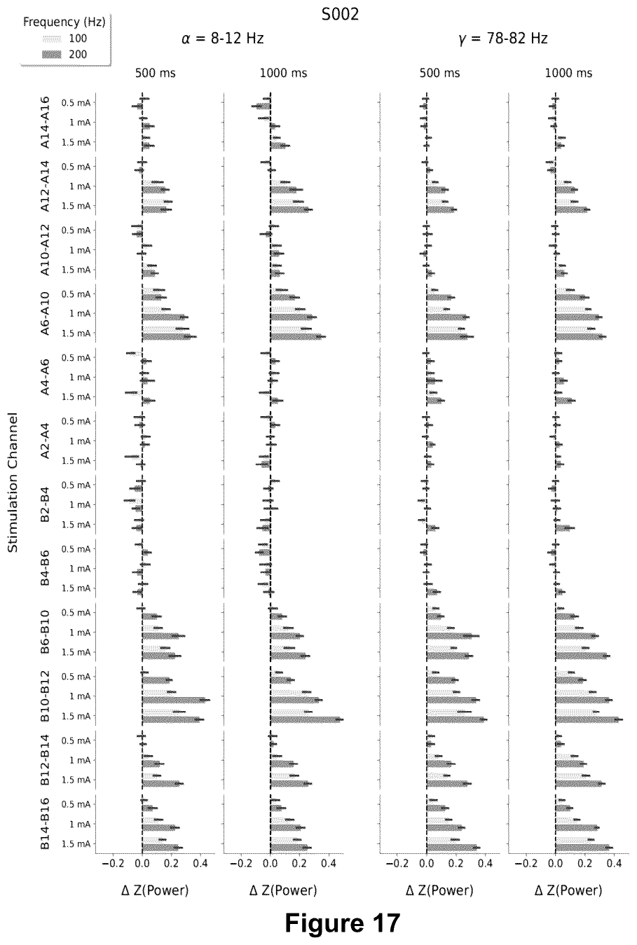

FIGS. 17 and 18 illustrate reveal considerable site-to-site heterogeneity in the effects of stimulation amplitude, frequency, and duration on alpha and gamma, in accordance with some embodiments. FIG. 17 reflects stimulation's physiological effects in Experiment 2. The effect of stimulation amplitude, frequency, duration, and location on alpha-band power (8-12 Hz, left panel) and gamma-band power (78-82 Hz, right panel) for animal S002. This analysis averages over the nine electrode pairs that do not overlap with either the anode or cathode. The effects of the stimulation parameters depend greatly on the location of stimulation. FIG. 18 reflects stimulation's physiological effects: in experiment 2. The effect of stimulation amplitude, frequency, duration, and location on alpha-band power (8-12 Hz, left panel) and gamma-band power (78-82 Hz, right panel) for animal S003. This analysis averages over the nine electrode pairs that do not overlap with either the anode or cathode. The effects of the stimulation parameters depend greatly on the location of stimulation.

In some embodiments, the site-to-site variability aligns with stimulation parameter search studies conducted in humans. Overall, stimulation-related power increases appeared far more prevalent than power decreases. To evaluate these effects, we fit linear mixed-effects models for each stimulation location in each animal (fixed effects: amplitude, frequency, duration, and pairwise interactions; random intercept for session; with predictors centered and normalized in Experiment 1). We FDR corrected across the parameters estimated within each subject and frequency band. For some stimulation locations (e.g., B10B12 in both animals), increasing stimulation amplitude and frequency led to greater increases in both alpha and gamma power (all t>8 and all p<0.001), mirroring Experiment 1. However, at other sites, we observe no effect of amplitude (e.g., A2-A4 in animal S002) or frequency (e.g., A4-A6 in animal S003) on alpha or gamma power. In some cases, we observe negative effects of amplitude (e.g., B4-B6 in animal S003, alpha and gamma power, both t<−4.0 and p<0.001) but we observe no negative effects of frequency.

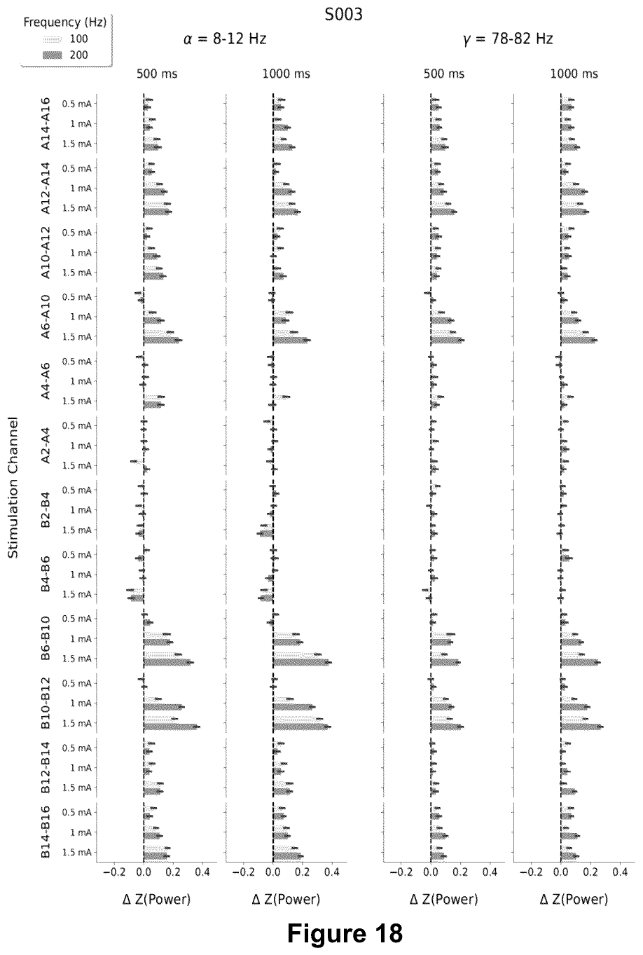

FIG. 19 illustrates linear mixed effects modeling of stimulation parameters, in accordance with some embodiments. Number of stimulation targets exhibiting significant effects of stimulation amplitude, frequency, duration (and all pairwise interactions) on changes in alpha and gamma-band power. Aggregated data from animals S002 and S003. More than half of all stimulation targets increased alpha and gamma power, and only two consistently decreased alpha power. Increasing stimulation amplitude tended to consistently increase post-stimulation changes in alpha and gamma; however, we did observe a small number of locations where changes in power decreased with increasing amplitude. Stimulation frequency exhibited a similar pattern to stimulation amplitude. We detected many fewer stimulation locations where duration reliably modulated either alpha or gamma power, and those locations did not show a clear directional pattern. The pairwise interactions between amplitude, frequency, and duration exhibited a diversity of results, mirroring that seen in FIGS. 17 and 18.

### 5. Closed Loop Modulation of Spectral Power

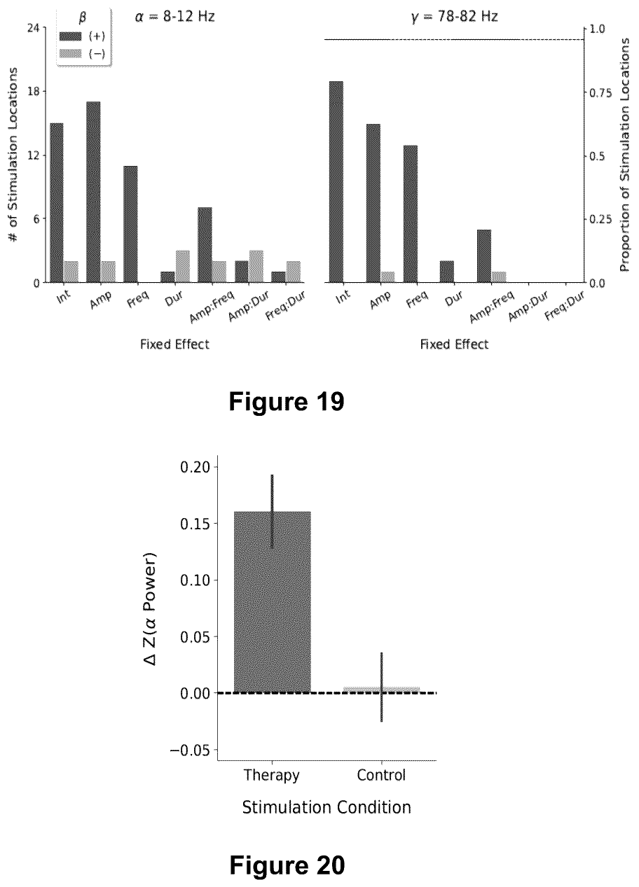

After demonstrating the system's capacity to record and decode neural signals and modulate spectral features with electrical stimulation, we tested its ability to perform these tasks in real time for closed-loop functionality. For S002, we selected a “therapy” stimulation parameter set from the parameter search data (θ_{T}) that reliably increased alpha-power and a “control” stimulation parameter set (θ_{C}) that did not change alpha-power. When the SNS detected low alpha-power (below the average value), we either stimulated with θ_{T} or θ_{C} and evaluated the subsequent changes in alpha-power. FIG. 20 illustrates that stimulation with the therapy parameter increased alpha-power in closed-loop; no such increase appeared for the control parameter set. Stimulation during periods of low alpha-power with a therapy parameter set reliably increased alpha-power, while a control parameter set did not modulate alpha-power. Error bars represent one standard error of the mean around average modulations from 57 sessions.

### 6. Brain Histology

We performed histopathology on brain tissue proximal to the implanted depth leads in all three animals. In brief, we fixated the brain, trimmed the region of interest, embedded it in paraffin, sectioned, and stained it with hematoxylin and eosin, Luxol Fast Blue (LFB) for demyelination, Glial Fibrillary Acidic Protein for astrocytosis, IBA-1 to identify macrophages and microglia, and Fluro Jade B (FJB) to evaluate neuronal necrosis. (FJB staining was performed in Animal 1 only.) The study pathologist utilized light microscopy to examine stained sections and assessed them based on predefined histological evaluation criteria for cellular and tissue response.

In some situations, no indicators of severe inflammatory response or damage were observed in any brain sections examined: (1) Inflammatory Cells: Polymorphonuclear cells, lymphocytes, multinucleated giant cells, and plasma cells (Score 0); and (2) Necrosis (Score 0). Observed changes in the primary brain tissue were consistent with minor injury resulting from the implant procedure itself: (1) Astrocytosis, defined as gliosis, was present, generally ranging from minimal to mild; (2) Expression of Iba-1, a marker for Microglia/Macrophage/Gitter Cells, ranged from minimal to moderate. (3) Demyelination was absent in nearly all sections examined across all three animals. (4) Neovascularization and Fibrosis were either absent or minimal (Score 1) in limited regions. In S001, which had the SNS Depth Lead implanted into the left hemisphere and a Control Lead implanted into the right hemisphere, we observed no significant differences in tissue response between the implanted brain hemispheres. While glial encapsulation of electrode arrays represents a concern for BCI systems that record single or multi-unit activity using high-impedance (~100 kΩ) micro-electrodes with a small surface area (~0.001 mm²), the SNS records local field potentials using low-impedance (<100Ω) macro-electrodes with a relatively large (5.3 mm²) surface area. These local field potentials are relatively immune to signal-to-noise changes due to glial encapsulation, which utilizes a similar electrode design to treat epilepsy using closed-loop neurostimulation and has demonstrated efficacy for more than nine years in many patients.

### 7. Additional Information

In some embodiments, Brain-responsive stimulation now represents an established therapy for epilepsy and movement disorders, with promise for treating chronic pain, depression, and other conditions. However, FDA-cleared platforms only decode a single spectral feature from a handful of channels. The Smart Neurostimulation System (SNS) overcomes this limitation by measuring electrical fields across 60 bipolar contacts on four depth leads and providing embedded algorithms for spectral processing and classifier-based stimulation control.

In some embodiments, the ability to stimulate the brain in response to distributed patterns of neural activity may be particularly useful for neurocognitive and affective disorders where functional impairments vary dramatically from moment to moment. In some embodiments, a closed-loop algorithm is deployed on a partially externalized device, showing that stimulating the lateral temporal cortex during predicted memory lapses produced significant memory improvements in sham-controlled, double-blinded studies of neurosurgical epilepsy patients. For instance, by training logistic regression classifiers on spectral activity during learning, these studies could reliably predict which items would be recalled or forgotten, then trigger stimulation during predicted memory lapses. Mnemonic benefits only appeared for closed-loop stimulation—random stimulation did not improve memory.

In some embodiments, we designed the SNS to deliver closed-loop stimulation chronically via an AI-enabled brain implant (IPG) that communicates wirelessly with an external processor (EP). The EP, modeled on a cochlear implant sound processor, delivers power inductively to the IPG and acts as a communication hub with the cloud-based AI platform. Clinical programmer software enables patient-specific models to trigger therapeutic stimulation based on real-time brain state variability. In memory applications, predicted memory lapses will trigger stimulation, and post-minus-pre-stimulation classifier output will guide stimulation parameter optimization.

In some embodiments, the SNS records field potentials and stimulate the brain in closed loop with onboard signal processing. The RNS records from a maximum of six channels in two brain regions compared with the SNS's 60 channels across four regions. Because seizures often localize to small brain regions, six channels significantly reduce seizures in many patients. However, memory-related brain signals occur across distributed networks, requiring the widespread surveillance capabilities of the SNS and the ability to adaptively select among a larger set of stimulation targets. Analyses of large open-data sets informed the SNS design by identifying whole-brain patterns of neural activity that signal periods of good and poor memory.

In some embodiments, a particular device records field potentials from each segmented STN or GPi lead, extracts a single β-band (13-30 Hz) power estimate once per second, and automatically raises or lowers stimulation amplitude when that biomarker crosses clinician-defined thresholds. In the pivotal trial, the algorithm maintained motor benefit while significantly reducing delivered charge. The particular device senses at most two bipolar channels and reacts to one spectral feature, making it unsuitable for decoding cognition functions whose neural features span widespread frequencies and distributed brain networks.

In some embodiments, an existing prior art system analyzes neural data and applies a control policy via an external computer rather than on the implanted device. This design tradeoff may limit the usability and portability of the existing prior art system, and achieve a more flexible and powerful algorithm development environment.

FIG. 13B is a table comparing characteristics of six example devices, in accordance with some embodiments, where Sym represents symmetric. SW represents square wave, FFT represents Fast Fourier Transform, IIR represents Infinite Impulse Response, and Stim. Lat. represents Stimulation Latency. Device 1 in FIG. 13B corresponds to an example SNS described in this application, and Devices 2 to 6 correspond to example alternative technologies that may be applied to implement neural stimulation or recording.

Our ovine study validates core SNS functionality. Due to the small sheep brain, we implanted two multi-contact depth leads. Stimulation at a single contact pair demonstrated that increasing amplitude and frequency systematically alter alpha and gamma-band activity at other recording electrodes. In some embodiments, personalized logistic regression classifiers reliably predicted animal movement in hold-out sessions, demonstrating in vivo that the SNS can detect and modulate behaviorally relevant neural signals. Histological analyses showed no adverse tissue response to either lead type.

In some embodiments, the ovine model, while valuable for demonstrating device functionality, has translation limitations. The smaller sheep brain necessitated only two shorter depth leads (with only nine bipolar pairs available for recording the effects of stimulation after excluding stimulation-adjacent contacts). Additionally, while our experiments demonstrated reliable decoding of movement and spectral power modulation, we did not exercise the SNS's ability to decode higher-cognitive functions, such as memory or its closed-loop functionality.

The Smart Neurostimulation System (SNS) represents a major advance in neuromodulatory technology, offering simultaneous 60-channel brain activity monitoring with automatically configurable, targeted electrical stimulation based on real-time spectral analysis of neural data. Ovine data demonstrate core functionality. Although designed to evaluate an earlier proof of-concept therapy for memory loss, the SNS's multi-channel sensing and brain-state-contingent stimulation holds the potential to address a broad range of pathologies that exhibit moment-to-moment fluctuations, such as depression, anxiety, chronic pain, and attention disorders. The platform's ability to record from distributed brain networks while delivering targeted stimulation to deep brain structures provides the foundation for a broad range of personalized, biomarker-guided therapies.

In some embodiments, the SNS 120 implements a closed-loop stimulation delivery using classifier-guided analysis that controls stimulation parameters dynamically and in real time during therapy. The stimulation parameters are customized and personalized in real time during therapy. Further, in some embodiments, a pre-stimulation classifier is applied to provide stimulation parameters (e.g., pre-stimulation classifier outputs), and while or after the first stimulation parameters are applied, a post-stimulation classifier is applied to update the stimulation parameters (e.g., to generate post-stimulation classifier outputs), thereby enhancing therapeutic effectiveness.

In some embodiments, the SNS 120 may analyze data collected from a patient, and extract patient-specific biomarkers (e.g., spectral decomposition, frequency-band power, and specific frequency ranges specific to the patient), e.g., using a machine learning model.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Additionally, it will be understood that, although the terms “first,” “second,” etc. may be used herein to describe various elements, these elements should not be limited by these terms. These terms are only used to distinguish one element from another.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

Although various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages can be implemented in hardware, firmware, software or any combination thereof.

What is claimed is:

1. A system for treating cognitive or neurological dysfunctions, comprising:
one or more electrode arrays including a plurality of electrodes, the one or more electrode arrays configured to be implanted in a brain region of a patient and provide a plurality of channels based on the plurality of electrodes;
an implantable electronics package coupled to the one or more electrode arrays, wherein the electronics package configured to be mounted on a skull under the scalp and including one or more processors and memory storing a cognitive computational model and one or more programs for execution by the one or more processors, wherein the one or more programs comprise instructions for:
concurrently collecting local field potential (LFP) recordings from the brain region of the patient via the plurality of channels provided by the one or more electrode arrays;
generating a spectral decomposition of the LFP recordings;
updating the cognitive computational model based on the spectral decomposition of the LFP recordings; and
based on the cognitive computational model, delivering stimulation to the brain region of the patient via at least a subset of the one or more electrode arrays; and
an external electronic system wearable by the patient, and configured to enable at least wireless data communication with the implantable electronics package.

2. The system of claim 1, wherein the external electronic system is disposed in proximity to the implantable electronics package and on top of the scalp, and power and data telemetry are provided to the implantable electronics package using a radio frequency (RF) coil system.

3. The system of claim 1, wherein the plurality of channels includes 30, 32, or 64 channels.

4. The system of claim 1, wherein the implantable electronics package further comprises:
a electronics module that is hermetically packaged, wherein the electronics module is configured to collect the LFP recordings and generate the stimulation without using a battery;
one or more coaxial RF coils configured to receive power from, and exchanging data with, the external electronic system; and
a subcortical probe configured to be implanted in the brain region of the patient.

5. The system of claim 1, wherein the patient is a human patient with one of a traumatic brain injury (TBI), Alzheimer's disease, epilepsy, major depression, attention-deficit hyperactivity disorder (ADHD), Parkinson's disease, and essential tremor.

6. The system of claim 1, wherein the implantable electronics package further includes a communication module configured to obtain the cognitive computational model from an external server.

7. The system of claim 1, wherein the external electronic system is configured to receive the cognitive computational model updated by the implantable electronics package and generate a plurality of predefined stimulation patterns based on the cognitive computational model, and wherein the stimulation is delivered based on the plurality of predefined stimulation patterns.

8. The system of claim 1, wherein delivering stimulation further comprises identifying at least the subset of the one or more electrode arrays corresponding to one or more brain areas associated with memory performance, wherein the stimulation is delivered to the one or more brain areas to activate the one or more brain areas.

9. The system of claim 1, wherein the one or more programs stored on the memory of the implantable electronics package further comprise instructions for:
processing the LFP recordings to generate processed LFP recordings; and
transmitting the processed LFP recordings to the external electronic system.

10. The system of claim 1, wherein delivering stimulation further comprises identifying at least the subset of the one or more electrode arrays that are implanted within at least one of a temporal lobe, a frontal lobe, a middle temporal gyrus, a left parietal lobe, a hippocampus, an entorhinal cortex, and a right parietal lobe of the brain region.

11. The system of claim 1, further comprising a computer system communicatively coupled to the external electronic system, wherein the computer system is configured to personalize the cognitive computational model for the patient and provide the cognitive computational model to the implantable electronics package via the external electronic system.

12. The system of claim 1, wherein the LFP recordings correspond to a moving sampling window, and the cognitive computational model represents samples in the moving sampling window with a respective number of features for each of the plurality of channels, the respective number being smaller than a predefined feature limit.

13. The system of claim 12, wherein:
the moving sampling window has a temporal length smaller than 800 millisecond;
the predefined feature limit is equal to 8;
a number of channels is less than or equal to 64;
a footprint of the implantable electronics package on the skull is less than 46 mm×37 mm; and
a power limit of the implantable electronics package is 25 mW or less.

14. The system of claim 1, wherein the external electronic system is configured to provide power to the implantable electronics package, and the power is lower than a power limit, and is wirelessly transmitted over the scalp by an inductive link coupled between the implantable electronics package and the external electronic system, and wherein the implantable electronics package includes a rechargeable configuration for storing the power.

15. The system of claim 1, wherein the one or more electrode arrays includes four electrode arrays, and each electrode array further has sixteen electrodes including a reference electrode.

16. The system of claim 1, wherein the subset of the one or more electrode arrays includes two electrodes on a first electrode array of the one or more electrode arrays, and delivering the stimulation further includes:
generating biphasic, square-wave, stimulation pulses; and
delivering the biphasic, square-wave, stimulation pulses in a bipolar configuration to the brain region of the patient via the two electrodes on the first electrode array.

17. The system of claim 16, wherein delivering the stimulation further comprises:
based on the cognitive computational model, determining a plurality of stimulation parameters including target electrode identifications, a pulse amplitude, a pulse width, and a stimulation duration.

18. The system of claim 1, the one or more programs further comprising instructions for:
applying a multivariate classifier to process the LFP recordings or the spectral decomposition to update the cognitive computational model, wherein the cognitive computational model includes a distribution of spectral power across electrodes, a subsequent recall status of encoded items, and a mapping between the distribution and the subsequent recall status, a spectral decomposition, a frequency-band power, and a plurality of target frequency ranges.

19. The system of claim 1, wherein the cognitive computational model includes a level of a mnemonic function, and the one or more programs further comprise instructions for one or more of:
applying a spectral filter associated with a frequency range to the LFP recordings;
estimating an LFP spectral power pattern for each of the plurality of channels at the frequency range; and
applying a machine learning model to process the LFP spectral power pattern and predict the level of the mnemonic function.

20. A method for treating cognitive or neurological dysfunction, comprising:
at a neuromodulation system including one or more electrode arrays, an implantable electronics package, and an external electronic system:
identifying a plurality of channels provided by a plurality of electrodes of the one or more electrode arrays, the one or more electrode arrays being implanted in a brain region of a patient;
collecting field potential (LFP) recordings from the brain region of the patient via the plurality of channels provided by the one or more electrode arrays;
generating a spectral decomposition of the LFP recordings by the implantable electronics package, the implantable electronics package is mounted on a skull under the scalp;
updating, by the implantable electronics package, a cognitive computational model based on the spectral decomposition of the LFP recordings; and
based on the cognitive computational model, delivering stimulation to the brain region of the patient via at least a subset of the one or more electrode arrays; and
enabling, by an external electronic system, at least wireless data communication with the implantable electronics package.
